(19) United States
(12) Patent Application Publication
(10) Pub. No.: US 2025/0073142 A1
(43) Pub. Date: Mar. 6, 2025

(54) Personal Care Compositions

(71) Applicant: Colgate-Palmolive Company, New York, NY (US)

(72) Inventors: Viktor DUBOVOY, Basking Ridge, NJ (US); Haroon Ahmed Chaudhry, Monroe Township, NJ (US); Emily Marr, Bridgewater, NJ (US); Ewelina Lesniak, North Brunswick, NJ (US); Hongwei Shen, Holmdel, NJ (US); Kristina Ivana Fabijanic, Metuchen, NJ (US); Andrei Potanin, Hillsborough, NJ (US); Baran Teoman, Union, NJ (US); Jonghun Lee, Metuchen, NJ (US); Matthew Cipriaso, Monroe Township, NJ (US)

(73) Assignee: Colgate-Palmolive Company, New York, NY (US)

(21) Appl. No.: 18/827,142

(22) Filed: Sep. 6, 2024

Related U.S. Application Data
(60) Provisional application No. 63/536,889, filed on Sep. 6, 2023.
(60) Provisional application No. 63/612,545, filed on Dec. 20, 2023.

(51) Int. Cl. A61K 8/31; A61K 8/04; A61K 8/36; A61K 8/37; A61K 8/64; A61K 8/67; A61K 8/891; A61Q 19/10

(52) U.S. Cl. CPC A61K 8/31; A61K 8/046; A61K 8/361; A61K 8/375; A61K 8/64; A61K 8/678; A61K 8/891; A61Q 19/10

(57) ABSTRACT

Personal care compositions are disclosed herein. In accordance with an aspect, provided is a personal care composition having a hydrocarbon having 4 to 6 carbons; a fatty ester, a fatty acid, a medium chain triglyceride, a silicone, vitamin E, or a combination of two or more thereof; and one or more surfactant, wherein the personal care composition is self-foaming.

### CROSS-REFERENCE TO RELATED APPLICATIONS

This application claims the benefit of priority from U.S. Patent Application No. 63/536,889, filed Sep. 6, 2023, titled Personal Care Compositions, and from U.S. Patent Application No. 63/612,545, filed Dec. 20, 2023, titled Personal Care Compositions, the content of both which is hereby incorporated herein in their entireties, for all purposes.

### BACKGROUND

Liquid cleansing compositions, such as aqueous foaming compositions like hand soaps, facial soaps or cleansers, body washes, and shower gels, should desirably have acceptable levels of foam production for consumer satisfaction. Foam production, for example, is a key attribute for consumers when choosing products. To achieve a desirable foam production, manufacturers incorporate sufficient quantities of foaming agents, such as surfactants, into the compositions.

However, in recent years, consumer demand for more natural products has increased—e.g., interest in the origin of raw materials and products with ingredients with a high degree of naturality. It is a challenge to produce formulations that have the physical characteristics that consumers expect, including desirable amounts of foam production and sufficient viscosity, while simultaneously achieving a high degree of naturality. Moreover, liquid consumer products normally contain different species of surfactants. Consequently, manufacturers are shifting to the use of natural/sustainable surfactant substitutes. One challenge with surfactant substitutes, however, is maintaining an acceptable foaming volume that is still acceptable for consumers.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In accordance with an aspect of the invention, provided is a personal care composition including a hydrocarbon having 4 to 6 carbons; a fatty acid; one or more surfactant, and wherein the personal care composition is self-foaming.

In accordance with another aspect of the invention, provided is a personal care composition including a hydrocarbon having 4 to 6 carbons; a silicone; one or more surfactant, and wherein the personal care composition is self-foaming.

In accordance with a further aspect of the invention, provided is a personal care composition including a hydrocarbon having 4 to 6 carbons; vitamin E; one or more surfactant, and wherein the personal care composition is self-foaming.

A non-limiting list of embodiments in accordance with certain aspects of the invention are disclosed below:

In accordance with an embodiment 1, provided is a personal care composition comprising:
a hydrocarbon having 4 to 6 carbons;
a fatty ester, a fatty acid, or a combination thereof;
one or more surfactant, and
wherein the personal care composition is self-foaming.

In accordance with an embodiment 2, provided is the personal care composition according to embodiment 1, wherein the hydrocarbon having 4 to 6 carbons is selected from a hydrocarbon having 5 carbons.

In accordance with an embodiment 3, provided is the personal care composition according to embodiment 1 or embodiment 2, wherein the hydrocarbon having 4 to 6 carbons comprises pentane, isopentane, or a combination thereof.

In accordance with an embodiment 4, provided is the personal care composition according to any foregoing embodiment, wherein the hydrocarbon having 4 to 6 carbons consists of pentane, isopentane, or a combination thereof.

In accordance with an embodiment 5, provided is the personal care composition according to any foregoing embodiment, wherein the fatty ester, the fatty acid, or the combination thereof comprises at least one fatty ester.

In accordance with an embodiment 6, provided is the personal care composition according to embodiment 5, wherein the fatty ester comprises a C₆-C₃₂ fatty ester.

In accordance with an embodiment 7, provided is the personal care composition according to embodiment 5, wherein the fatty ester is selected from cetyl ester, cetearyl octanoate, isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, octyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, dicaprylyl carbonate, pentaerythritol esters, polyglyceryl-10 oleate, polyglyceryl-10 dioleate, polyglyceryl-6 stearate, polyglyceryl-6 distearate, polyglyceryl-10 stearate, polyglyceryl-10 distearate, polyglyceryl-8 dipalmitate, polyglyceryl-10 dipalmitate, polyglyceryl-10 behenate, polyglyceryl-12 trilaurate, ethylhexyl isononanoate, and a combination of two or more thereof.

In accordance with an embodiment 8, provided is the personal care composition according to one of embodiment 5 to embodiment 7, wherein the fatty ester comprises octyl isononanoate, isononyl isononanoate, isopropyl myristate, ethylhexyl isononanoate, or a combination thereof.

In accordance with an embodiment 9, provided is the personal care composition according to any foregoing embodiment, wherein the fatty ester, the fatty acid, or the combination thereof comprises at least one fatty acid.

In accordance with an embodiment 10, provided is the personal care composition according to embodiment 9, wherein the fatty acid comprises a C₆-C₃₂ fatty acid, such as a C₈-C₁₈ fatty acid.

In accordance with an embodiment 11, provided is the personal care composition according to embodiment 9, wherein the fatty acid comprises octanoic acid, lauric acid, palmitic acid, stearic acid, behenic acid, arachidonic acid, oleic acid, isostearic acid, polyhydroxystearic acid, sebacic acid, coconut acid, or a combination of two or more thereof.

In accordance with an embodiment 12, provided is the personal care composition according to one of embodiment 9 to embodiment 11, wherein the fatty acid comprises octanoic acid, stearic acid, polyhydroxystearic acid, coconut acid, or a combination of two or more thereof.

In accordance with an embodiment 13, provided is the personal care composition according to any foregoing embodiment, wherein the personal care composition has a weight ratio of the total amount of hydrocarbon having 4 to 6 carbons to the total amount of the fatty ester, the fatty acid, or the combination thereof of about 2:1 to about 1:7.

In accordance with an embodiment 14, provided is the personal care composition according to any foregoing embodiment, wherein the personal care composition comprises a fatty acid selected from octanoic acid, stearic acid, polyhydroxystearic acid, and a combination of two or more thereof, and a fatty ester selected from octyl isononanoate, isononyl isononanoate, isopropyl myristate, and a combination of two or more thereof.

In accordance with an embodiment 15, provided is the personal care composition according to embodiment 14, wherein the personal care composition has a weight ratio of the total amount of the hydrocarbon having 4 to 6 carbons to the total amount of the fatty ester to the total amount of fatty acid from about 2:1:1 to about 1:5:5.

In accordance with an embodiment 16, provided is the personal care composition according to any foregoing embodiment, wherein the one or more surfactant comprises an acyl amino acid surfactant.

In accordance with an embodiment 17, provided is the personal care composition according to embodiment 16, wherein the acyl amino acid surfactant is derived from alanine, arginine, aspartic acid, glutamic acid, glycine, isoleucine, leucine, lysine, phenylalanine, serine, tyrosine, valine, sarcosine, threonine, or taurine.

In accordance with an embodiment 18, provided is the personal care composition according to embodiment 16 or embodiment 17, wherein the acyl amino acid surfactant is selected from sodium cocoyl glycinate, potassium cocoyl glycinate, N-lauroyl-N-methyltaurine, sodium cocoyl amino acids, lysine lauroyl methionate, N-stearoyl-l-leucine, N-stearoyl-l-glutamic acid, a salt thereof, and a combination of two or more thereof.

In accordance with an embodiment 19, provided is the personal care composition according to any foregoing embodiment, wherein the one or more surfactant comprises an anionic surfactant of plant protein.

In accordance with an embodiment 20, provided is the personal care composition according to embodiment 19, wherein the anionic surfactant of plant protein is selected from sodium cocoyl hydrolyzed keratin, sodium cocoyl hydrolyzed rice protein, sodium cocoyl apple amino acid, sodium cocoyl hydrolyzed soy protein, sodium cocoyl hydrolyzed sweet almond protein, sodium cocoyl hydrolyzed wheat protein, sodium cocoyl hydrolyzed wheat protein glutamate, sodium cocoyl oat amino acids, sodium cocoyl/palmoyl/sunfloweroyl glutamate, potassium lauroyl wheat amino acid, sodium lauroyl wheat amino acid, sodium lauroyl oat amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof.

In accordance with an embodiment 21, provided is the personal care composition according to embodiment 19 or embodiment 20, wherein the anionic surfactant of plant protein is selected is selected from potassium lauroyl wheat amino acid, sodium lauroyl wheat amino acid, sodium lauroyl oat amino acid, sodium cocoyl apple amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof.

In accordance with an embodiment 22, provided is the personal care composition according to any foregoing embodiment, wherein the one or more surfactant comprises an anionic surfactant of silk protein.

In accordance with an embodiment 23, provided is the personal care composition according to embodiment 22, wherein the anionic surfactant of silk protein comprises sodium cocoyl hydrolyzed silk.

In accordance with an embodiment 24, provided is the personal care composition according to any foregoing embodiment, wherein the one or more surfactant comprises a nonionic surfactant.

In accordance with an embodiment 25, provided is the personal care composition according to embodiment 24, wherein the nonionic surfactant comprises a lactylate surfactant.

In accordance with an embodiment 26, provided is the personal care composition according to embodiment 25, wherein the lactylate surfactant is selected from sodium decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, and a combination of two or more thereof.

In accordance with an embodiment 27, provided is the personal care composition according to one of embodiment 24 to embodiment 26, wherein the nonionic surfactant comprises an alkyl glucoside.

In accordance with an embodiment 28, provided is the personal care composition according to embodiment 27, wherein the alkyl glucoside is selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination of two or more thereof.

In accordance with an embodiment 29, provided is the personal care composition according to any foregoing embodiment, wherein the one or more surfactant is a surfactant system.

In accordance with an embodiment 30, provided is the personal care composition according to any foregoing embodiment further comprising water in an amount from about 10 to 90 wt. %, based on the total weight of the personal care composition.

In accordance with an embodiment 31, provided is the personal care composition according to any foregoing embodiment further comprising a polyol.

In accordance with an embodiment 32, provided is the personal care composition according to embodiment 31, wherein the polyol is selected from glycerine, 1,3-propanediol, isoprene glycol, pentylene glycol, hexylene glycol, ethylene glycol, propylene glycol, butylene glycol, diethylene glycol, dipropylene glycol, diglycerol, erythritol, arabitol, adonitol, sorbitol, dulcitol, glucose, fructose, xylose, trehalose, sucrose, maltose, saccharose, lactose, and a combination of two or more thereof.

In accordance with an embodiment 33, provided is the personal care composition according to any foregoing embodiment, wherein the personal care composition is substantially free of or free of methyl perfluoroisobutyl ether and methyl perfluorobutyl ether.

In accordance with an embodiment 34, provided is the personal care composition according to any foregoing embodiment, wherein the personal care composition is substantially free of or free of polyfluoroalkyl substances.

In accordance with an embodiment 35, provided is a personal care product comprising:
a container containing a personal care composition according to any foregoing embodiment, wherein the personal care product is not pressurized within the container.

In accordance with an embodiment 36, provided is a personal care product comprising:
a container containing a personal care composition, the personal care composition comprising:
   a hydrocarbon having five carbons;
   a fatty ester, a fatty acid, or a combination thereof; and
   a surfactant system comprising:
      an anionic surfactant, and
      nonionic surfactant,
wherein the personal care composition is self-foaming, and wherein the personal care product is not pressurized.

In accordance with an embodiment 37, provided is a personal care composition comprising:
a hydrocarbon having 4 to 6 carbons;
a fatty acid;
one or more surfactant, and
wherein the personal care composition is self-foaming.

In accordance with an embodiment 38, provided is the personal care composition according to embodiment 37 further comprising a vitamin E or a derivative thereof.

In accordance with an embodiment 39, provided is the personal care composition according to embodiment 37 or embodiment 38, wherein the fatty acid comprises a C₆-C₃₂ fatty acid.

In accordance with an embodiment 40, provided is the personal care composition according to one of embodiment 37 to embodiment 39, wherein the fatty acid is selected from coconut acid, octanoic acid, lauric acid, palmitic acid, stearic acid, behenic acid, arachidonic acid, oleic acid, isostearic acid, polyhydroxystearic acid, sebacic acid, caproic acid, caprylic acid, capric acid, undecylic acid, lauric acid, myristic acid, pentadecylic acid, palmitic acid, and a combination of two or more thereof.

In accordance with an embodiment 41, provided is the personal care composition according to one of embodiment 37 to embodiment 40, wherein the fatty acid is selected from coconut acid, octanoic acid, stearic acid, polyhydroxystearic acid, and a combination of two or more thereof.

In accordance with an embodiment 42, provided is the personal care composition according to one of embodiment 37 to embodiment 41, wherein the personal care composition has a weight ratio of the total amount of hydrocarbon having 4 to 6 carbons to the total amount of the fatty acid of about 4:1 to about 1:5.

In accordance with an embodiment 43, provided is the personal care composition according to one of embodiment 37 to embodiment 42, wherein the fatty acid is present in an amount from about 0.5 to about 10 wt. %, based on the total weight of the personal care composition.

In accordance with an embodiment 44, provided is a personal care composition comprising:
a hydrocarbon having 4 to 6 carbons;
a silicone;
one or more surfactant, and
wherein the personal care composition is self-foaming.

In accordance with an embodiment 45, provided is the personal care composition according to embodiment 44 further comprising a vitamin E.

In accordance with an embodiment 46, provided is the personal care composition according to embodiment 44 or embodiment 45, wherein the silicone is selected from disiloxane, polydimethicone, dimethiconol, amodimethicone, aminoethylaminopropyl dimethicone, aminopropyl dimethicone, trimethylsilylamodimethicone, trisiloxane, tetrasiloxane, and a combination of two or more thereof.

In accordance with an embodiment 47, provided is the personal care composition according to one of embodiment 44 to embodiment 46, wherein the silicone is selected from disiloxane, polydimethicone, and a combination thereof.

In accordance with an embodiment 48, provided is the personal care composition according to one of embodiment 44 to embodiment 47, wherein the personal care composition has a weight ratio of the total amount of hydrocarbon having 4 to 6 carbons to the total amount of the silicone of about 4:1 to about 1:5.

In accordance with an embodiment 49, provided is the personal care composition according to one of embodiment 44 to embodiment 48, wherein the silicone is present in an amount from about 0.5 to about 10 wt. %, based on the total weight of the personal care composition.

In accordance with an embodiment 50, provided is a personal care composition comprising:
a hydrocarbon having 4 to 6 carbons;
vitamin E;
one or more surfactant, and
wherein the personal care composition is self-foaming.

In accordance with an embodiment 51, provided is the personal care composition according to embodiment 50 further comprising a fatty acid, a silicone, or a combination of two or more thereof.

In accordance with an embodiment 52, provided is the personal care composition according to embodiment 50 or embodiment 51, wherein the vitamin E is selected from alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopheryl phosphate, and a combination of two or more thereof.

In accordance with an embodiment 53, provided is the personal care composition according to one of embodiment 50 to embodiment 52, wherein the vitamin E comprises alpha-tocopherol.

In accordance with an embodiment 54, provided is the personal care composition according to one of embodiment 50 to embodiment 53, wherein the personal care composition has a weight ratio of the total amount of hydrocarbon having 4 to 6 carbons to the total amount of the vitamin E of about 8:1 to about 1:1.

In accordance with an embodiment 55, provided is the personal care composition according to one of embodiment 50 to embodiment 54, wherein the vitamin E is present in an amount from about 0.1 to about 7 wt. %, based on the total weight of the personal care composition.

In accordance with an embodiment 56, provided is the personal care composition according to one of embodiment 37 to embodiment 55, wherein the hydrocarbon having 4 to 6 carbons is selected from a hydrocarbon having 5 carbons.

In accordance with an embodiment 57, provided is the personal care composition according to one of embodiment 37 to embodiment 56, wherein the hydrocarbon having 4 to 6 carbons comprises pentane, isopentane, or a combination thereof.

In accordance with an embodiment 58, provided is the personal care composition according to one of embodiment 37 to embodiment 57, wherein the hydrocarbon having 4 to 6 carbons consists of pentane, isopentane, or a combination thereof.

In accordance with an embodiment 59, provided is the personal care composition according to one of embodiment 37 to embodiment 58, wherein the one or more surfactant comprises an acyl amino acid surfactant.

In accordance with an embodiment 60, provided is the personal care composition according to embodiment 59, wherein the acyl amino acid surfactant is derived from alanine, arginine, aspartic acid, glutamic acid, glycine, isoleucine, leucine, lysine, phenylalanine, serine, tyrosine, valine, sarcosine, threonine, or taurine.

In accordance with an embodiment 61, provided is the personal care composition according to embodiment 59 or embodiment 60, wherein the acyl amino acid surfactant is selected from sodium cocoyl glycinate, potassium cocoyl glycinate, N-lauroyl-N-methyltaurine, sodium cocoyl amino acids, lysine lauroyl methionate, N-stearoyl-1-leucine, N-stearoyl-1-glutamic acid, a salt thereof, and a combination of two or more thereof.

In accordance with an embodiment 62, provided is the personal care composition according to one of embodiment 37 to embodiment 61, wherein the one or more surfactant comprises an anionic surfactant of plant protein.

In accordance with an embodiment 63, provided is the personal care composition according to embodiment 62, wherein the anionic surfactant of plant protein is selected from sodium cocoyl hydrolyzed keratin, sodium cocoyl hydrolyzed rice protein, sodium cocoyl apple amino acid, sodium cocoyl hydrolyzed soy protein, sodium cocoyl hydrolyzed sweet almond protein, sodium cocoyl hydrolyzed wheat protein, sodium cocoyl hydrolyzed wheat protein glutamate, sodium cocoyl oat amino acids, sodium cocoyl/palmoyl/sunfloweroyl glutamate, potassium lauroyl wheat amino acid, sodium lauroyl wheat amino acid, sodium lauroyl oat amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof.

In accordance with an embodiment 64, provided is the personal care composition according to embodiment 62 or embodiment 63, wherein the anionic surfactant of plant protein is selected from potassium lauroyl wheat amino acid, sodium lauroyl wheat amino acid, sodium lauroyl oat amino acid, sodium cocoyl apple amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof.

In accordance with an embodiment 65, provided is the personal care composition according to one of embodiment 37 to embodiment 64, wherein the one or more surfactant comprises an anionic surfactant of silk protein.

In accordance with an embodiment 66, provided is the personal care composition according to embodiment 65, wherein the anionic surfactant of silk protein comprises sodium cocoyl hydrolyzed silk.

In accordance with an embodiment 67, provided is the personal care composition according to one of embodiment 37 to embodiment 66, wherein the one or more surfactant comprises a nonionic surfactant.

In accordance with an embodiment 68, provided is the personal care composition according to embodiment 67, wherein the nonionic surfactant comprises a lactylate surfactant.

In accordance with an embodiment 69, provided is the personal care composition according to embodiment 68, wherein the lactylate surfactant is selected from sodium decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, and a combination of two or more thereof.

In accordance with an embodiment 70, provided is the personal care composition according to one of embodiment 67 to embodiment 69, wherein the nonionic surfactant comprises an alkyl glucoside.

In accordance with an embodiment 71, provided is the personal care composition according to embodiment 70, wherein the alkyl glucoside is selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination of two or more thereof.

In accordance with an embodiment 72, provided is the personal care composition according to one of embodiment 37 to embodiment 71, wherein the one or more surfactant comprises a surfactant system.

In accordance with an embodiment 73, provided is the personal care composition according to one of embodiment 37 to embodiment 72 further comprising water in an amount from about 10 to 90 wt. %, based on the total weight of the personal care composition.

In accordance with an embodiment 74, provided is the personal care composition according to any one of embodiment 37 to embodiment 73 further comprising a polyol.

In accordance with an embodiment 75, provided is the personal care composition according to embodiment 74, wherein the polyol is selected from glycerin, 1,3-propanediol, isoprene glycol, pentylene glycol, hexylene glycol, ethylene glycol, propylene glycol, butylene glycol, diethylene glycol, dipropylene glycol, diglycerol, erythritol, arabitol, adonitol, sorbitol, dulcitol, glucose, fructose, xylose, trehalose, sucrose, maltose, saccharose, lactose, and a combination of two or more thereof.

In accordance with an embodiment 76, provided is the personal care composition according to one of embodiment 37 to embodiment 75, wherein the personal care composition is substantially free of or free of methyl perfluoroisobutyl ether and methyl perfluorobutyl ether.

In accordance with an embodiment 38, provided is the personal care composition according of one of embodiment 37 to embodiment 76, wherein the personal care composition is substantially free of or free of polyfluoroalkyl compounds.

In accordance with an embodiment 78, provided is a personal care product comprising:
a container containing a personal care composition according to one of embodiment 37 to embodiment 77, wherein the personal care product is not pressurized within the container.

In accordance with an embodiment 79, provided is a personal care composition according to any foregoing embodiment further comprising a triglyceride having an alkyl chain comprised of 6 to 10 carbons.

In accordance with an embodiment 80, provided is a personal care composition comprising:
a hydrocarbon having 4 to 6 carbons;
a fatty ester, a fatty acid, a triglyceride having an alkyl chain comprised of 6 to 10 carbons, a silicone, vitamin E, or a combination of two or more thereof;
one or more surfactant, and
wherein the personal care composition is self-foaming.

In accordance with an embodiment 81, provided is a personal care composition according to embodiment 80, wherein the surfactant system comprises sodium cocoyl glycinate.

In accordance with an embodiment 82, provided is a personal care composition according to embodiment 81, wherein the sodium cocoyl glycinate is present in an amount from about 0.5 to about 6 wt. %.

In accordance with an embodiment 83, provided is a personal care composition according to embodiment 82, wherein the amount of sodium cocoyl glycinate is from about 1.1 to about 1.65 wt. %.

In accordance with an embodiment 84, provided is a personal care composition according to embodiment 82, wherein the amount of sodium cocoyl glycinate is from about 1.6 to 6 wt. %.

In accordance with an embodiment 85, provided is a personal care composition according to any one of embodiment 80 to embodiment 84, wherein the surfactant system further comprises an alkyl amino acid surfactant.

In accordance with an embodiment 86, provided is a personal care composition according to embodiment 85, wherein the alkyl amino acid surfactant comprises an alkali metal cocoyl glutamate.

In accordance with an embodiment 87, provided is a personal care composition according to any one of embodiment 80 to embodiment 86, wherein the surfactant system further comprises an anionic sulfonate surfactant.

In accordance with an embodiment 88, provided is a personal care composition according to embodiment 86, wherein the anionic sulfonate surfactant comprises olefin sulfonate surfactants, such as sodium C₁₄-C₁₆ olefin sulfonate surfactant and potassium C₁₄-C₁₆ olefin sulfonate surfactants, sodium tridecyl benzene sulfonate, sodium dodecyl benzene sulfonate, or a combination thereof.

In accordance with an embodiment 89, provided is a personal composition according to one of embodiments 80 to 83 and 85 to 88, wherein the surfactant system comprises a total amount of sodium cocoyl glycinate and/or potassium cocoyl glycinate of about 0.1 to about 1.65 wt. % and at least one of an acyl amino acid surfactant other than sodium cocoyl glycinate or potassium cocoyl glycinate.

In accordance with an embodiment 90, provided is a personal composition according to one of embodiments 80 to 83 and 85 to 89, wherein the surfactant system consists of a total amount of sodium cocoyl glycinate and/or potassium cocoyl glycinate of about 0.1 to about 1.65 wt. % and at least one of an acyl amino acid surfactant other than sodium cocoyl glycinate or potassium cocoyl glycinate, a sulfonate surfactant (e.g. an alkali metal olefin sulfonate surfactant), an anionic taurate surfactant, an anionic glutamate surfactant, an anionic isethionate surfactant, a betaine surfactant, a glucoside surfactant, or a combination of two or more thereof.

In accordance with an embodiment 91, provided is a personal composition according to one of embodiments 80 to 83 and 85 to 90, wherein the surfactant system consists of a total amount of sodium cocoyl glycinate and/or potassium cocoyl glycinate of about 0.1 to about 1.65 wt. % and at least one surfactant selected from the group consisting of an acyl amino acid surfactant other than sodium cocoyl glycinate or potassium cocoyl glycinate, a sulfonate surfactant (e.g. an alkali metal olefin sulfonate surfactant), an anionic taurate surfactant, an anionic glutamate surfactant, an anionic isethionate surfactant, a betaine surfactant, a glucoside surfactant, and a combination of two or more thereof.

In accordance with an embodiment 92, provided is the personal care composition according to any foregoing embodiment, wherein the personal care composition is formulated to produce a foam height of about 1 mm or more, preferably about 1.1 mm or more, or preferably about 1.2 mm or more, e.g., within 30 seconds of being rubbed in a circular motion about 16 times at a rate of about 96 bpm and on a heated substrate at a temperature of about 37° C.

In accordance with an embodiment 93, provided is a personal care composition according to any foregoing embodiment, wherein the personal care composition further comprises one or more particulate(s) selected from the group consisting of fumed silica, calcined clay, rice starch, silica (e.g., hydrated silica), and a combination of two or more thereof.

In accordance with an embodiment 94, provided is a personal care composition according to any one of embodiment 80 to embodiment 93, wherein the personal care composition comprises one or more triglyceride(s), wherein the one or more triglyceride(s) consists of medium chain triglycerides. In other words, embodiment 92 is substantially free of or free of triglycerides other than medium chain triglycerides.

In accordance with an embodiment 95, provided is a personal care composition according to any foregoing embodiment, wherein the personal care composition is formulated to produce a first foam height of about 1 mm or more within 30 seconds after applied to a heated substrate at a temperature of about 37° C. and rubbed in a circular motion about 16 times at a rate of 96 rubs per minute and produce a second foam height within 80 seconds of said being rubbed in a circular motion, wherein the second foam height is at least 45% greater than the first foam height.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a bar graph of the foam volume increase during mixing for non-limiting example compositions and a comparative composition in accordance with aspects of the invention;

FIG. 2 is a bar graph showing the maximum foam height produced during mixing for non-limiting example compositions and a comparative composition in accordance with aspects of the invention;

FIG. 3 is a graph of the rate of vertical foam growth as a function of time for non-limiting compositions in accordance with aspects of the invention; and

FIG. 4 is a bar graph of the average maximum foam height for non-limiting compositions in accordance with aspects of the invention.

It should be understood that the various aspects are not limited to the compositions, arrangements, and instrumentality shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “hr”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

“Volatile”, as used herein, means having a flash point of less than about 100° C. “Non-volatile”, as used herein, means having a flash point of greater than about 100° C.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

Any member in a list of species that is used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that defines or exemplifies the genus is open, and it is given that other species may exist that define or exemplify the genus just as well as, or better than, any other species listed.

The phrases, “a mixture thereof,” “a combination thereof,” or “a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the instant disclosure can be free or essentially free of all components and elements positively recited throughout the instant disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both a polysaccharide and a thickener. If a particular personal care composition includes and/or recites both a polysaccharide and a thickener, the compound that may be characterized as both a polysaccharide and a thickener will serve only as either the polysaccharide or the thickener—not both.

For readability purposes, the chemical functional groups are in their adjective form; for each of the adjectives, the word “group” is assumed. For example, the adjective “alkyl” without a noun thereafter, should be read as “an alkyl group.”

Aspects of the invention relate to personal care compositions, including personal care compositions that are self-foaming, e.g., after being dispersed from a container and/or being applied by a user. The inventors discovered that personal care composition may be formulated to be self-forming by, inter alia, include certain ingredients in particular amounts and/or ratios. For instance, in certain preferred embodiments, the personal care composition includes certain amounts of a hydrocarbon having 4 to 6 carbons and certain fatty acids, fatty esters silicones, and/or vitamin E in particular amounts and weight ratios.

In accordance with an aspect of the invention, provided is a personal care composition including a hydrocarbon having 4 to 6 carbons; a fatty ester, a fatty acid, a medium chain triglyceride, a silicone, vitamin E, or a combination of two or more thereof; one or more surfactant, and wherein the personal care composition is self-foaming.

In accordance with another aspect of the invention, provided is a personal care composition including a hydrocarbon having 4 to 6 carbons; a silicone; one or more surfactant, and wherein the personal care composition is self-foaming.

In accordance with a further aspect of the invention, provided is a personal care composition including a hydrocarbon having 4 to 6 carbons; vitamin E; one or more surfactant, and wherein the personal care composition is self-foaming.

In accordance with yet a further aspect of the invention, provided is a personal care product comprising: a container containing a personal care composition, the personal care composition comprising: a hydrocarbon having 4 to 6 carbons; a fatty ester, a fatty acid, a medium chain triglyceride, a silicone, vitamin E, or a combination of two or more thereof; and a surfactant system, wherein the personal care composition is self-foaming, and wherein the personal care product is not pressurized.

In accordance with another aspect of the invention, provided is a personal care product comprising a container containing a personal care composition, the personal care composition comprising a surfactant system comprising an anionic surfactant, and nonionic surfactant a hydrocarbon having five carbons, and a fatty ester, a fatty acid, or a combination thereof, wherein the personal care composition is self-foaming, and wherein the personal care product is not pressurized.

The personal care compositions may have a reduced amount of, be substantially free of, or free of methyl perfluoroisobutyl ether and methyl perfluorobutyl ether. For instance, in some embodiments, the personal care composition comprises about 3 wt. % or less, about 2 wt. % or less, about 1 wt. % or less, about 0.5 wt. % or less, about 0.1 wt. % or less, about 0.05 wt. % or less, or about 0.01 wt. % or less of methyl perfluoroisobutyl ether and/or methyl perfluorobutyl ether, based on the total weight of the personal care composition. In at least one embodiment, the personal care composition contains about 0 wt. % or 0 wt. % of methyl perfluoroisobutyl ether and/or methyl perfluorobutyl ether, based on the total weight of the personal care composition.

Additionally or alternatively, the personal care compositions may have a reduced amount of, be substantially free of, or free of polyfluoroalkyl compounds (PFAS). For instance, in some embodiments, the personal care composition comprises about 3 wt. % or less, about 2 wt. % or less, about 1 wt. % or less, about 0.5 wt. % or less, about 0.1 wt. % or less, about 0.05 wt. % or less, or about 0.01 wt. % or less of polyfluoroalkyl compounds (PFAS), based on the total weight of the personal care composition. In at least one embodiment, the personal care composition contains 0 wt. % of polyfluoroalkyl compounds (PFAS), based on the total weight of the personal care composition.

The personal care compositions may be substantially free of or free of magnesium ion sources, such as magnesium silicate. In some cases, the total amount of magnesium ion sources, such as magnesium silicate, is about 0.1 wt. % or less, about 0.05 wt. % or less, about 0.01 wt. % or less, about 0.005 wt. % or less, or about 0.001 wt. % or less, based on the total weight of the personal care composition.

Suitable components, such as those listed below, may be included or excluded from the formulations for the personal care compositions depending on the specific combination of other ingredients, the form of the personal care composition, and/or the use of the composition (e.g., cleanser (such as, a facial cleanser), a face wash, a body wash, a serum, a lotion, a cream, a spray, etc.). For example, the personal care composition may include a hydrophilic phase and a hydrophobic phase when the personal care composition is in the form of an emulsion or a two-phase composition. In some embodiments, the personal care composition is in the form of a skin care composition, a cleansing compositions, and/or a facial cleansing composition. Preferably, the personal care composition is self-foaming. The personal care composition may be referred to a as self-foaming compositions herein when the personal care composition is self-foaming. The personal care composition should not be interpreted as not-self foaming when the personal care composition is simply referred to as a personal care composition.

The personal care compositions are typically self-foaming compositions. The personal care compositions may foam upon being dispersed from the container containing such personal care composition and/or upon application to a keratin substrate, such as skin. For example, the personal care composition may foam and/or start to foam within 1 second, 2 seconds, 4 seconds, 6 seconds, 10 seconds, 15 seconds, 30 seconds, or 60 seconds from being dispersed from the container. Preferably, the personal care composition foam is capable of foaming without the use of mechanical foaming apparatuses, such as a pump with a foaming chamber. Additionally or alternatively, the personal care composition may foam within 1 second, 2 seconds, 4 seconds, 6 seconds, 10 seconds, 15 seconds, 30 seconds, or 60 seconds from being applied to a keratin substrate, such as skin.

The personal care composition may be formulated to produce a max foam height of about 1 mm or more, about 1.1 mm or more, about 1.2 mm or more, about 1.4 mm or more, about 1.6 mm or more, about 1.7 mm or more, about 1.8 mm or more, about 1.9 mm or more, or about 2 mm or more after being applied to a substrate. In some embodiments, the personal care composition is formulated to produce a max foam height of about 1 mm or more, about 1.1 mm or more, about 1.2 mm or more, about 1.4 mm or more, about 1.6 mm or more, about 1.7 mm or more, about 1.8 mm or more, about 1.9 mm or more, or about 2 mm or more after being applied to a substrate (e.g. skin) having a temperature of about 37° C.

The personal care composition may be further rubbed on a keratin substrate to, in some instances, increase foaming of the personal care composition. For example, the personal care composition may be formulated such that when applied to a heated substrate (e.g., skin) at a temperature of, e.g., about 37° C., and rubbed in a circular motion about 16 times at a rate of 96 rubs per minute, the personal care composition produces a first foam height of about 1 mm or more at or within 30 seconds after being rubbed and produces a second foam height that is at least 75% greater than the first foam height at or within 80 seconds after said being rubbed. In some embodiments, the personal care composition is formulated to produce a first foam height at or within 30 seconds after being applied to a heated substrate (e.g., skin) at a temperature of, e.g., about 37° C., and rubbed in a circular motion about 16 times at a rate of 96 rubs per minute of about 1 mm or more, about 1.1 mm or more, about 1.2 mm or more, about 1.4 mm or more, about 1.6 mm or more, about 1.7 mm or more, about 1.8 mm or more, about 1.9 mm or more, or about 2 mm or more. Additionally, the personal care composition may be formulated to produce a second foam height at or within 80 seconds after being applied to a heated substrate (e.g., skin) at a temperature of, e.g., about 37° C., and rubbed in a circular motion about 16 times at a rate of 96 rubs per minute, wherein the second foam height greater than the first foam height by at least 45%, at least 55%, at least 65%, at least 75%, at least 80%, at least 85%, at least 90%, at least 95%, at least 100%, at least 105%, or at least 110%.

It was surprisingly discovered that the above foam heights may achieved by personal care compositions after undergoing accelerated aging by kept in a controlled environment at a temperature of 40° C. for 4 weeks. Additionally or alternatively, the personal care compositions may be specifically adapted to achieve an average maximum height according to DFA lather performance after being after undergoing accelerated aging by kept in a controlled environment at a temperature of 40° C. for 4 weeks of about 70 mL or more, about 74 mL or more, about 77 mL or more, about 79 mL or more, about 82 mL or more, or about 85 mL or more.

The personal care composition typically comprises one or more hydrocarbon(s) comprising from 4 to 6 carbons (C₄₋₆ hydrocarbons). The total amount of C₄₋₆ hydrocarbon(s) may be from about 0.5 to about 10 wt. %, based on the total weight of the personal care composition. For example, the personal care composition may contain C₄₋₆ hydrocarbon(s) in an amount from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1.5 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %, about 1 to about 1.75 wt. %; from about 1.5 to about 10 wt. %, about 1.5 to about 8 wt. %, about 1.5 to about 6 wt. %, about 1.5 to about 5 wt. %, about 1.5 to about 4 wt. %, about 1.5 to about 3 wt. %, about 1.3 to about 2 wt. %, about 1.5 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 9 wt. %, or any range or subrange thereof, based on the total weight of the personal care composition.

The C₄₋₆ hydrocarbon(s) may comprise hydrocarbons having 4 carbons, 5 carbons, 6 carbons, or any range or combination thereof. In at least one preferred embodiment, the C₄₋₆ hydrocarbon(s) comprises a hydrocarbon having 5 carbons (C₅ hydrocarbon(s)). For example, the C₅ hydrocarbon(s) may comprise pentane, isopentane, or a combination thereof. In certain embodiments, the C₄₋₆ hydrocarbon(s) consists of C₅ hydrocarbon(s). The self-foaming personal care composition may comprise isopentane in an amount up to about 1.5 wt. %, up to about 1.25 wt. %, up to about 1 wt. %; from about 0.1 to about 1.5 wt. %, about 0.1 to about 1.25 wt. %, about 0.1 to about 1 wt. %; from about 0.5 to about 1.5 wt. %, about 0.5 to about 1.25 wt. %, about 0.5 to about 1 wt. %, or any range or subrange thereof, based on the total weight of the self-foaming personal care composition.

The self-foaming personal care composition may, preferably, comprise isopentane and pentane in a weight ratio of isopentane to pentane of about 4:1 to about 1:3, about 3:1 to about 1:3, about 2:1 to about 1:3, about 1:1 to about 1:3; from about 4:1 to about 1:2, about 3:1 to about 1:2, about 2:1 to about 1:2, about 1:1 to about 1:2; from about 4:1 to about 1:1, about 3:1 to about 1:1, about 2:1 to about 1:1, or any range or subrange thereof.

The inventors discovered that self-foaming personal care compositions comprising a C₄₋₆ hydrocarbon(s) can unexpectedly be improved by incorporating certain combinations of fatty ester(s), fatty acid(s), silicone(s), triglycerides (e.g., medium chain triglycerides), and vitamin E in particular amounts and weight ratios thereof. One such benefit achieved by the personal care compositions disclosed herein is a reduced foaming during the production process of such personal care composition. It was unexpectedly discovered, however, that certain combinations of fatty ester(s), fatty acid(s), silicone(s), triglycerides (e.g., medium chain triglycerides), and vitamin E in particular amounts and weight ratios can achieve both a reduced amount of foaming during the production process of such personal care composition while maintaining desirable levels of foaming during use (e.g., lather) of the personal care composition. Further, it was discovered that certain surfactant system in conjunction with the combinations of fatty ester(s), fatty acid(s), silicone(s), triglycerides, and vitamin E can enhance the self-foaming characteristic of the personal care composition, especially after aging.

In certain embodiments, the personal care compositions include one or more fatty acid(s). The personal care composition may include an amount of fatty acid(s) ranging from about 0.5 to about 10 wt. %, based on the total weight of the personal care composition. For example, the total amount of the one or more fatty acid(s) may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1.5 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 1.5 to about 10 wt. %, about 1.5 to about 8 wt. %, about 1.5 to about 6 wt. %, about 1.5 to about 5 wt. %, about 1.5 to about 4 wt. %, about 1.5 to about 3 wt. %, about 1.5 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 9 wt. %, or any range or subrange thereof, based on the total weight of the personal care composition.

The one or more fatty acid(s) that may be present in the personal care composition may be straight or branched chain acids and/or may be saturated or unsaturated. The fatty acid may be selected from C₆-C₃₂ fatty acids, such as C₈-C₃₂ fatty acids, C₁₀-C₃₂ fatty acids, C₁₂-C₃₂ fatty acids, C₆-C₂₄ fatty acids, C₆-C₂₂ fatty acids, C₁₀-C₂₄ fatty acids, C₁₀-C₂₂ fatty acids, C₁₂-C₂₄ fatty acids, C₁₂-C₂₂ fatty acids, a C₆-C₂₂ fatty acid, a C₆-C₁₈ fatty acid, or a C₈-C₁₈ fatty acid. In some embodiments, the one or more fatty acid(s) may consist of those selected from a C₈-C₁₈ fatty acid. Non-limiting examples of fatty acids include diacids, triacids, and other multiple acids as well as salts of these fatty acids. For example, the fatty acid may optionally include or be chosen from octanoic acid, lauric acid, palmitic acid, stearic acid, behenic acid, arachidonic acid, oleic acid, isostearic acid, polyhydroxystearic acid, sebacic acid, caproic acid, caprylic acid, capric acid, undecylic acid, lauric acid, myristic acid, pentadecylic acid, palmitic acid and a combination of two or more thereof. In some embodiments, the fatty acids are selected from the group consisting of stearic acid, isostearic acid, polyhydroxystearic acid, octanoic acid, and a combination of two or more thereof. In further embodiments, the fatty acids are selected from the group consisting of caproic acid, caprylic acid, capric acid, undecylic acid, lauric acid, myristic acid, pentadecylic acid, palmitic acid, and a combination of two or more thereof. Additional examples of fatty acids include arachidic acid, arachidonic acid, beeswax acid, capric acid, caproic acid, caprylic acid, coconut acid, isostearic acid, lauric acid, linoleic acid, linolenic acid, myristic acid, oleic acid, olive acid, palmitic acid, rapeseed acid, stearic acid, tallow acid, undecanoic acid, undecylenic acid, and wheat germ acid. In at least one preferred embodiment, the fatty acid comprises coconut acid and polyhydroxystearic acid.

The personal care compositions may be formulated to have a weight ratio of the total amount of C₄₋₆ hydrocarbons to the total amount of fatty acid(s) of about 4:1 to about 1:5, about 3:1 to about 1:5, about 2:1 to about 1:5, about 1:1 to about 1:5; from about 4:1 to about 1:4, about 3:1 to about 1:4, about 2:1 to about 1:4, about 1:1 to about 1:4; from about 4:1 to about 1:3, about 3:1 to about 1:3, about 2:1 to about 1:3, about 1:1 to about 1:3; from about 4:1 to about 1:2, about 3:1 to about 1:2, about 2:1 to about 1:2, about 1:1 to about 1:2; from about 4:1 to about 1:1, about 3:1 to about 1:1, about 2:1 to about 1:1, or about 1:1.

Additionally or alternatively, the personal care composition may include one or more silicone. The amount of silicone in the personal care composition may be from about 0.5 to about 10 wt. %, based on the total weight of the personal care composition. For example, the total amount of the one or more silicone(s) may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1.5 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 1.5 to about 10 wt. %, about 1.5 to about 8 wt. %, about 1.5 to about 6 wt. %, about 1.5 to about 5 wt. %, about 1.5 to about 4 wt. %, about 1.5 to about 3 wt. %, about 1.5 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 9 wt. %, or any range or subrange thereof, based on the total weight of the personal care composition.

The silicone may be selected from selected from disiloxane, polydimethicone, dimethiconol, amodimethicone, aminoethylaminopropyl dimethicone, aminopropyl dimethicone, trimethylsilylamodimethicone, trisiloxane, tetrasiloxane, and a combination of two or more thereof. In certain cases, the silicone is selected from disiloxane, polydimethicone, and a combination thereof.

In some embodiments, the silicone may be chosen from the polydialkylsiloxanes, such as the polydimethylsiloxanes (PDMS), and the organo-modified polysiloxanes having at least one functional group chosen from the poly(alkoxylated) groups, the amine groups and the alkoxy groups. Examples of organomodified silicones include polydiaryl siloxanes, such as polydiphenylsiloxanes, and polyalkyl-arylsiloxanes. Among the organomodified silicones, non-limiting mention can be made of the polyorganosiloxanes.

The personal care compositions may, preferably, be formulated to have a weight ratio of the total amount of C₄₋₆ hydrocarbons to the total amount of silicone(s) of about 4:1 to about 1:5. For example, the personal care composition may have a weight ratio of the total amount of C₄₋₆ hydrocarbons to the total amount of silicone of about 3:1 to about 1:5, about 2:1 to about 1:5, about 1:1 to about 1:5; from about 4:1 to about 1:4, about 3:1 to about 1:4, about 2:1 to about 1:4, about 1:1 to about 1:4; from about 4:1 to about 1:3, about 3:1 to about 1:3, about 2:1 to about 1:3, about 1:1 to about 1:3; from about 4:1 to about 1:2, about 3:1 to about 1:2, about 2:1 to about 1:2, about 1:1 to about 1:2; from about 4:1 to about 1:1, about 3:1 to about 1:1, about 2:1 to about 1:1, or about 1:1.

The personal care composition may comprise triglycerides, preferably a medium chain triglyceride. In some embodiments, the triglyceride(s) consists of medium chain triglycerides. The personal care composition may be free of or substantially free of triglycerides other than medium triglyceride(s). The total amount of triglyceride(s) in the personal care composition may be from about 0.1 to about 7 wt. %, based on the total weight of the personal care composition. In some instances, the total amount of triglyceride(s) in the personal care composition may be from about 0.1 to about 6 wt. %, about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 7 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 7 wt. %, about 1.5 to about 6 wt. %, about 1.5 to about 5 wt. %, about 1.5 to about 4 wt. %, about 1.5 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 4 to about 5 wt. %; from about 5 to about 7 wt. %, about 5 to about 6 wt. %, or any range or subrange thereof, based on the total weight of the personal care composition.

Medium chain triglycerides, as used herein, refers to a triglyceride having at least one aliphatic carbon chain comprised of 6 to 10 carbons. For example, a medium chain triglyceride may have one, two, or three aliphatic carbons chains of 6 to 10 carbons, e.g., 6 to 9 carbons, 6 to 8 carbons, 7 to 10 carbons, 7 to 9 carbons, or 8 to 10 carbons. In some embodiments, the medium chain triglyceride has one, two, or three aliphatic carbon chain of 6 carbons, 7 carbons, 8 carbons, 9 carbons, and/or 10 carbons. In at least one embodiment the medium chain triglyceride comprises or consists of triheptanoin.

In certain embodiments, the personal care composition may, additionally or alternatively, include a vitamin E. The vitamin E may be present in the personal care composition in an amount from about 0.1 to about 7 wt. %, based on the total weight of the personal care composition. In some instances, the total amount of vitamin E in the personal care composition may be from about 0.1 to about 6 wt. %, about 0.1 to about 5 wt. %, about 0.1 to about 4 wt. %, about 0.1 to about 3 wt. %, about 0.1 to about 2 wt. %; from about 0.3 to about 5 wt. %, about 0.3 to about 4 wt. %, about 0.3 to about 3 wt. %, about 0.3 to about 2 wt. %; from about 0.6 to about 5 wt. %, about 0.6 to about 4 wt. %, about 0.6 to about 3 wt. %, about 0.6 to about 2 wt. %; from about 0.9 to about 5 wt. %, about 0.9 to about 4 wt. %, about 0.9 to about 3 wt. %, about 0.9 to about 2 wt. %; from about 1.2 to about 7 wt. %, about 1.2 to about 6 wt. %, about 1.2 to about 5 wt. %, about 1.2 to about 4 wt. %, about 1.2 to about 3 wt. %, about 1.2 to about 2 wt. %; from about 1.5 to about 7 wt. %, about 1.5 to about 6 wt. %, about 1.5 to about 5 wt. %, about 1.5 to about 4 wt. %, about 1.5 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 4 to about 5 wt. %; from about 5 to about 7 wt. %, about 5 to about 6 wt. %, or any range or subrange thereof, based on the total weight of the personal care composition.

Non-limiting examples of vitamin E include alpha-tocopherol, beta-tocopherol, gamma-tocopherol, delta-tocopherol, alpha-tocotrienol, beta-tocotrienol, gamma-tocotrienol, delta-tocotrienol, tocopheryl acetate, tocopherol phosphate, natural extracts enriched in vitamin E derivatives, or combinations thereof. The vitamin E may be selected from alpha-tocopherol, beta-tocopherol, gamma-tocopherol, delta-tocopherol, alpha-tocotrienol, beta-tocotrienol, gamma-tocotrienol, delta-tocotrienol, tocopheryl acetate, tocopherol phosphate, and a combination of two or more thereof. In some instances, the personal care composition comprises alpha-tocopherol.

In some preferred embodiments, the personal care composition is formulated to have a weight ratio of the total amount of C₄₋₆ hydrocarbons to the total amount of vitamin E of about 8:1 to about 1:1. For example, the personal care composition may have a weight ratio of the total amount of C₄₋₆ hydrocarbons to the total amount of vitamin E of about 7:1 to about 1:1, about 6:1 to about 1:1, about 5:1 to about 1:1, about 4:1 to about 1:1, about 3:1 to about 1:1; from about 8:1 to about 1:1, about 7:1 to about 1:1, about 6:1 to about 1:1, about 5:1 to about 1:1, about 4:1 to about 1:1, about 3:1 to about 1:1; from about 8:1 to about 2:1, about 7:1 to about 2:1, about 6:1 to about 2:1, about 5:1 to about 2:1, about 4:1 to about 2:1, about 3:1 to about 2:1; from about 8:1 to about 3:1, about 7:1 to about 3:1, about 6:1 to about 3:1, about 5:1 to about 3:1, about 4:1 to about 3:1; from about 8:1 to about 4:1, about 7:1 to about 4:1, about 6:1 to about 4:1, about 5:1 to about 4:1; from about 8:1 to about 5:1, about 7:1 to about 5:1, about 6:1 to about 5:1; from about 8:1 to about 6:1, about 7:1 to about 6:1, or any range or subrange thereof.

In addition to a fatty acid, a silicone, a triglyceride, and/or vitamin E, the personal care composition may include one or more fatty ester. The one or more fatty ester may be selected from C₆-C₃₂ fatty esters, such as C₈-C₃₂ fatty esters, C₁₀-C₃₂ fatty esters, C₁₂-C₃₂ fatty esters, C₆-C₂₄ fatty esters, C₆-C₂₂ fatty esters, C₁₀-C₂₄ fatty esters, C₁₀-C₂₂ fatty esters, C₁₂-C₂₄ fatty esters, or C₁₂-C₂₂ fatty esters. The fatty ester may be straight or branched chain acids and/or may be saturated or unsaturated. The fatty ester may be a selected from cetyl ester, purcellin oil (cetearyl octanoate), isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, octyl isononanoate, ethylhexyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, dicaprylyl carbonate, pentaerythritol esters, and a combination of two or more thereof. Other fatty esters worth mentioning include polyglyceryl-10 oleate, polyglyceryl-10 dioleate, polyglyceryl-6 stearate, polyglyceryl-6 distearate, polyglyceryl-10 stearate, polyglyceryl-10 distearate, polyglyceryl-8 dipalmitate, polyglyceryl-10 dipalmitate, polyglyceryl-10 behenate, polyglyceryl-12 trilaurate, and a combination of two or more thereof.

The one or more fatty ester can be selected from dialkyl carbonates of formula: R₁O(C═O)R₂, wherein R₁ and R₂ are independently linear or branched, saturated or unsaturated alkyl chains having 1 to 30 carbon atoms, or having 2 to 28 carbon atoms, or having 4 to 25 carbon atoms, or having 6 to 22 carbon atoms, preferably one or more fatty carbonates selected from C₁₄₋₁₅ dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, and a mixture thereof.

Non-limiting examples of polyglycerol esters of fatty acids include those of the following formula:

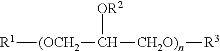

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀.

In certain embodiments, the personal care composition may comprise octyl isononanoate, isononyl isononanoate, ethylhexyl isononanoate, isopropyl myristate, or a combination thereof. Additionally or alternatively, in certain embodiments, the personal care composition may comprise coconut acid and/or a salt thereof.

The fatty ester(s) may be present in the personal care composition in an amount from about 0.5 to about 10 wt. %, based on the total weight of the personal care composition. For example, the total amount of the one or more fatty ester(s) may be from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1.5 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 1.5 to about 10 wt. %, about 1.5 to about 8 wt. %, about 1.5 to about 6 wt. %, about 1.5 to about 5 wt. %, about 1.5 to about 4 wt. %, about 1.5 to about 3 wt. %, about 1.5 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 9 wt. %, or any range or subrange thereof, based on the total weight of the personal care composition.

In certain embodiments, the personal care composition includes at least one fatty acid and at least one fatty ether. For example, the personal care composition may comprises a fatty acid selected from octanoic acid, stearic acid, polyhydroxystearic acid, and a combination of two or more thereof, and a fatty ester selected from octyl isononanoate, isononyl isononanoate, isopropyl myristate, and a combination of two or more thereof.

Additionally or alternatively, the personal care composition may be formulated to have a weight ratio of the total amount of hydrocarbon having 4 to 6 carbons (e.g., pentane, isopentane, etc.) to the total amount of the fatty ester to the total amount of the fatty acid from about 2:1:1 to about 1:5:5. For example, the personal care composition may have a weight ratio of the total amount of the hydrocarbon having 4 to 6 carbons to the total amount of the fatty ester to the total amount of the fatty acid of about 2:1:1 to about 1:5:5, about 2:1:1 to about 1:4:4, about 2:1:1 to about 1:3:3, about 2:1:1 to about 1:2:2, about 2:1:1 to about 1:1:1; from about 1:1:1 to about 1:5:5, about 1:1:1 to about 1:4:4, about 1:1:1 to about 1:3:3, about 1:1:1 to about 1:2:2, or any range or subrange thereof.

In certain embodiments, the personal care composition may be formulated to have a weight ratio of the total amount of hydrocarbon having 4 to 6 carbons (e.g., pentane, isopentane, etc.) to the total amount of the fatty acid to the total amount of the vitamin E from about 8:4:1 to about 1:1:1. For example, the weight ratio of the total amount of hydrocarbon having 4 to 6 carbons (e.g., pentane, isopentane, etc.) to the total amount of the fatty acid to the total amount of the vitamin E may be from about 6:3:1 to about 1:1:1, about 4:2:1 to about 1:1:1, about 3:1.5:1 to about 1:1:1; from about 6:3:1 to about 2:1.25:1, about 4:2:1 to about 2:1.25:1, about 3:1.5:1 to about 2:1.25:1; from about 6:3:1 to about 3:1.5:1, about 4:2:1 to about 3:1.5:1, or any range or subrange thereof.

The personal care composition may be formulated to have a weight ratio of the total amount of hydrocarbon having 4 to 6 carbons (e.g., pentane, isopentane, etc.) to the total amount of the silicone to the total amount of the vitamin E from about 9:3:1 to about 1:1:1. For example, the weight ratio of the total amount of hydrocarbon having 4 to 6 carbons (e.g., pentane, isopentane, etc.) to the total amount of the silicone to the total amount of the vitamin E from about 9:3:1 to about 1:1:1, about 7:2:1 to about 1:1:1, about 5:1:1 to about 1:1:1, about 3:1:1 to about 1:1:1; about 2:1:1 to about 1:1:1; from about 9:3:1 to about 2:1:1, about 7:2:1 to about 2:1:1, about 5:1:1 to about 2:1:1, about 3:1:1 to about 2:1:1; from about 9:3:1 to about 4:1:1, about 7:2:1 to about 4:1:1, about 5:1:1 to about 4:1:1; from about 9:3:1 to about 4:2:1, about 7:2:1 to about 4:2:1 or any range or subrange thereof.

As noted above, the personal care composition may be formulated to include certain amounts of the one or more hydrocarbon having 4 to 6 carbons and certain amounts of fatty acid, silicone, vitamin E, fatty ester, medium chain triglyceride, or any combinations thereof in particular weight ratios. For instance, the personal care composition may be formulated to have a weight ratio of the total amount of hydrocarbon having 4 to 6 carbons (e.g., pentane, isopentane, etc.) to the total amount of the fatty acid, silicone, vitamin E, fatty ester, medium chain triglyceride, or any combinations thereof of about 2:1 to about 1:7. In certain embodiments, the personal care composition may preferably have a weight ratio of the total amount of hydrocarbon having 4 to 6 carbons to the total amount of the fatty acid, silicone, vitamin E, fatty ester, medium chain triglyceride, or any combinations thereof of about 2:1 to about 1:6, about 2:1 to about 1:5, about 2:1 to about 1:4, about 2:1 to about 1:3, about 2:1 to about 1:2, about 2:1 to about 1:1; from about 1:1 to about 1:7, about 1:1 to about 1:6, about 1:1 to about 1:5, about 1:1 to about 1:4, about 1:1 to about 1:3, about 1:1 to about 1:2; from about 1:2 to about 1:7, about 1:2 to about 1:6, about 1:2 to about 1:5, about 1:2 to about 1:4, about 1:2 to about 1:3; from about 1:3 to about 1:7, about 1:3 to about 1:6, about 1:3 to about 1:5, about 1:3 to about 1:4; from about 1:4 to about 1:7, about 1:4 to about 1:6, about 1:4 to about 1:5, or any range or subrange thereof.

As mentioned above, the inventors unexpectedly discovered that certain combinations of fatty ester(s), fatty acid(s), silicone(s), triglycerides (e.g., medium chain triglycerides), and vitamin E in particular amounts and weight ratios can achieve both a reduced amount of foaming during the production process of such personal care composition while maintaining desirable levels of foaming during use (e.g., lather) of the personal care composition. For example, certain embodiments of the personal care composition include one or more fatty acid (e.g., a fatty acid comprising or consisting of octanoic acid, lauric acid, palmitic acid, stearic acid, behenic acid, arachidonic acid, oleic acid, isostearic acid, polyhydroxystearic acid, sebacic acid, and/or coconut acid); one or more fatty ester (e.g., a fatty ester comprising or consisting of octyl isononanoate, isononyl isononanoate, and/or isopropyl myristate); one or more silicone (e.g., a silicone comprising or consisting of disiloxane, polydimethicone, dimethiconol, amodimethicone, aminoethylaminopropyl dimethicone, aminopropyl dimethicone, trimethylsilylamodimethicone, trisiloxane, and/or tetrasiloxane); one or more triglycerides (preferably, a medium chain triglycerides, such as a medium chain triglyceride having alkyl chain of 6 to 9 carbons); vitamin E (e.g., a vitamin E comprising or consisting of alpha-tocopherol, beta-tocopherol, gamma-tocopherol, delta-tocopherol, alpha-tocotrienol, beta-tocotrienol, gamma-tocotrienol, delta-tocotrienol, tocopherol acetate, and/or tocopheryl phosphate); or a combination of two or more thereof.

Preferably, personal care composition includes one or more fatty acid, such as those selected from octanoic acid, lauric acid, palmitic acid, stearic acid, behenic acid, arachidonic acid, oleic acid, isostearic acid, polyhydroxystearic acid, sebacic acid, and/or coconut acid; one or more fatty ester, such as those selected from octyl isononanoate, isononyl isononanoate, and/or isopropyl myristate; and at least one of a silicone (e.g., a silicone selected from disiloxane, polydimethicone, dimethiconol, amodimethicone, aminoethylaminopropyl dimethicone, aminopropyl dimethicone, trimethylsilylamodimethicone, trisiloxane, tetrasiloxane, and a combination thereof), a triglyceride (e.g., triglycerides selected from medium chain triglycerides), and/or a vitamin E (e.g., a vitamin E selected from alpha-tocopherol, beta-tocopherol, gamma-tocopherol, delta-tocopherol, alpha-tocotrienol, beta-tocotrienol, gamma-tocotrienol, delta-tocotrienol, tocopherol acetate, tocopheryl phosphate, and a combination thereof). In some embodiments, the personal care composition includes one or more fatty acid, one or more fatty ester, and one or more silicone, such as those described above. In further embodiments, the personal care composition includes one or more fatty acid, one or more fatty ester, and one or more triglyceride, such as those described above. In additional embodiments, the personal care composition includes one or more fatty acid, one or more fatty ester, and one or more vitamin E, such as those described above. In yet further embodiments, the personal care composition includes one or more silicone, one or more vitamin E, and optionally one or more triglyceride, such as those described above.

Additionally or alternatively, the personal care composition may comprise vitamin B3 compound, preferably in an effective amount. The amount of vitamin B3 in the personal care composition may be from vitamin B3. For example, the vitamin B3 may be present in an amount from about 0.1 to about 4 wt. %, based on the total weight of the personal care composition. In some embodiments, the vitamin B3 is present in the personal care composition in amount from about 0.1 to about 4 wt. %, about 0.1 to about 3 wt. %, about 0.1 to about 2 wt. %, about 0.1 to about 1 wt. %; from about 0.1 to about 4 wt. %, about 0.1 to about 3 wt. %, about 0.1 to about 2 wt. %, about 0.1 to about 1 wt. %; from about 0.1 to about 4 wt. %, about 0.1 to about 3 wt. %, about 0.1 to about 2 wt. %, about 0.1 to about 1 wt. %; from about 0.2 to about 4 wt. %, about 0.2 to about 3 wt. %, about 0.2 to about 2 wt. %, about 0.2 to about 1 wt. %; from about 0.5 to about 4 wt. %, about 0.5 to about 3 wt. %, about 0.5 to about 2 wt. %, about 0.5 to about 1 wt. %; from about 0.7 to about 4 wt. %, about 0.7 to about 3 wt. %, about 0.7 to about 2 wt. %, about 0.7 to about 1 wt. %; from about 1 to about 4 wt. %, about 1 to about 3 wt. %, about 1 to about 2 wt. %; from about 1.5 to about 4 wt. %, about 1.5 to about 3 wt. %, about 1.5 to about 2 wt. %; from about 2 to about 4 wt. %, about 2 to about 3 wt. %; from about 2.5 to about 4 wt. %, about 2.5 to about 3 wt. %, about 3 to about 4 wt. %, or any range or subrange thereof, based on the total weight of the personal care composition.

The vitamin B3 may selected from compounds having the following formula:

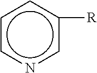

wherein R is —CONH2 (i.e., niacinamide), —COOH (i.e., nicotinic acid) or —CH2OH (i.e., nicotinyl alcohol); a derivative thereof, and/or a salt thereof. Examples of the vitamin B3 is selected from the group consisting of nicotinic acid esters, non-vasodilating esters of nicotinic acid, nicotinyl amino acids, nicotinyl alcohol esters of carboxylic acids (e.g., straight-chain or branched chain, cyclic or acyclic, saturated or unsaturated (including aromatic), and substituted or unsubstituted, nicotinic acid N-oxide and niacinamide N-oxide, tocopherol nicotinate and inositol hexanicotinate. Examples of derivatives of niacinamide include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., C1-C18). Specific examples of such derivatives include nicotinuric acid and nicotinyl hydroxamic acid. Additional derivatives of niacinamide includes nicotinyl alcohol esters, such as nicotinyl alcohol esters of the carboxylic acids, salicylic acid, acetic acid, glycolic acid, palmitic acid and the like Further examples of derivatives of niacinamide include 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine, and combinations thereof. In at least one preferred embodiment, the personal care composition includes vitamin B3.

The personal care composition may include a fatty compound selected from fatty compounds other than fatty acids, fatty esters, and triglycerides, such as fatty alcohols, fatty ethers, and/or oils. For example, fatty compounds may include or be chosen from one or more oil(s). The term “oil” means any fatty substance that is in liquid form at room temperature (24-25° C.) and at atmospheric pressure (760 mmHg). The oil may be a hydrocarbon-based oil, such as a saturated hydrocarbon oil, an unsaturated hydrocarbon oil, a lipid, a natural oil, and/or a synthetic oil. The one or more oil(s) may be synthetic oils and/or plant based oils. For instance, the oil may be a synthetic oil, such as a mineral oil and/or an alkane (paraffins). Light (low viscosity) highly branched hydrocarbon oils may also be suitable in some instances. In some embodiments, the oil is selected from plant based or vegetable based oils, such as coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, and a mixture thereof.

Examples of fatty alcohols that may be incorporated into certain embodiments of the personal care composition include those having a fatty group with a carbon chain of greater than 8 carbon atoms, 8 to 50 carbon atoms, 8 to 40 carbon atoms, 8 to 30 carbon atoms, 8 to 22 carbon atoms, 12 to 22 carbon atoms, or 12 to 18 carbon atoms, including all ranges and subranges therebetween. In some instances, the fatty group of the fatty alcohols has a carbon chain of 10 to 20 carbon atoms or 10 to 18 carbon atoms. The fatty alcohols may be chosen from polyethylene glycol ethers, such as those having a fatty alcohol group with a carbon chain of 12 to 16 or 12 to 14 carbon atoms.

The fatty alcohol may be hydrogenated (e.g., stearyl, lauryl, cetyl, and cetearyl) or may contain one or more double bonds (e.g., oleyl). Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a mixture thereof. In some cases, the fatty alcohols comprise at least one of or may be chosen from myristyl alcohol, lauryl alcohol, cetyl alcohol, stearyl alcohol, cetearyl alcohol, isostearyl alcohol, oleyl alcohol, isotridecyl alcohol, and a mixture thereof.

The fatty alcohol may be saturated or unsaturated. Exemplary saturated liquid fatty alcohols may be branched and optionally contain in their structure at least one aromatic or non-aromatic ring. In some instances, the fatty alcohols are acyclic. Non-limiting examples of liquid saturated fatty alcohols include octyldodecanol, isostearyl alcohol, and 2-hexyldecanol.

Exemplary unsaturated liquid fatty alcohol may include in their structure at least one double or triple bond. For example, the fatty alcohols may include several double bonds (such as 2 or 3 double bond), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol, and undecylenyl alcohol.

The amount of fatty compounds other than fatty ester(s), fatty acids(s), and triglyceride(s) may be from about 0.5 to about 10 wt. %, about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1.5 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; from about 1.5 to about 10 wt. %, about 1.5 to about 8 wt. %, about 1.5 to about 6 wt. %, about 1.5 to about 5 wt. %, about 1.5 to about 4 wt. %, about 1.5 to about 3 wt. %, about 1.5 to about 2 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %, about 3 to about 4 wt. %; from about 4 to about 10 wt. %, about 4 to about 8 wt. %, about 4 to about 6 wt. %, about 4 to about 5 wt. %; from about 5 to about 10 wt. %, about 5 to about 8 wt. %, about 5 to about 6 wt. %; from about 7 to about 10 wt. %, about 7 to about 9 wt. %, or any range or subrange thereof, based on the total weight of the personal care composition.

The personal care compositions typically comprise one or more surfactant, e.g., in an amount from about 1 to about 20 wt. %, based on the total weight of the personal care composition. In some instances, the total amount of the one or more surfactant(s) may be about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 6 wt. %, about 1 to about 4 wt. %; from about 2 to about 20 wt. %, about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 6 wt. %, about 2 to about 4 wt. %; from about 3 to about 20 wt. %, about 3 to about 18 wt. %, about 3 to about 16 wt. %, about 3 to about 14 wt. %, about 3 to about 12 wt. %, about 3 to about 10 wt. %, about 3 to about 8 wt. %, about 3 to about 6 wt. %, about 3 to about 5 wt. %; from about 4 to about 20 wt. %, about 4 to about 18 wt. %, about 4 to about 16 wt. %, about 4 to about 14 wt. %, about 4 to about 12 wt. %, about 4 to about 10 wt. %, about 4 to about 8 wt. %, about 4 to about 6 wt. %; form about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 7 to about 20 wt. %, about 7 to about 18 wt. %, about 7 to about 16 wt. %, about 7 to about 14 wt. %, about 7 to about 12 wt. %, about 7 to about 10 wt. %, about 5 to about 8 wt. %; from about 10 to about 20 wt. %, about 10 to about 18 wt. %, about 10 to about 16 wt. %, about 10 to about 14 wt. %, about 7 to about 12 wt. %; from about 13 to about 20 wt. %, about 13 to about 18 wt. %, about 13 to about 16 wt. %, or any range or subrange thereof, based on the total weight of the personal care composition.

The personal care compositions may include a surfactant system comprising two or more surfactants. The surfactant system typically includes two or more surfactants selected from non-sulfate based anionic surfactants, amino acid surfactants, amphoteric surfactants, nonionic surfactants, and combinations of two or more thereof. For example, the surfactant system may include two, three, four, five, six, seven, eight, nine surfactants, or any range or subrange thereof.

The one or more surfactant(s) may include an acyl amino acid surfactant. The acyl amino acid surfactant may be anionic. The acyl amino acid surfactant may be derived from alanine, arginine, aspartic acid, glutamic acid, glycine, isoleucine, leucine, lysine, phenylalanine, serine, tyrosine, valine, sarcosine, threonine, and/or taurine. The cation associated with the acyl amino acid surfactant can be sodium or potassium. Alternatively, the cation can be an organic salt, such as triethanolamine (TEA) or a metal salt. For example, the acyl amino acid surfactant may be selected from sodium cocoyl glycinate, potassium cocoyl glycinate, disodium cocoyl glutamate, potassium cocoyl glutamate, n-lauroyl-n-methyltaurine, sodium cocoyl amino acids, lysine lauroyl methionate, n-stearoyl-l-leucine, n-stearoyl-l-glutamic acid, a salt thereof, and a combination of two or more thereof. The surfactant system may comprise two or more acyl amino acid surfactant comprising or consisting of sodium cocoyl glycinate, potassium cocoyl glycinate, disodium cocoyl glutamate, potassium cocoyl glutamate, n-lauroyl-n-methyltaurine, sodium cocoyl amino acids, lysine lauroyl methionate, n-stearoyl-l-leucine, n-stearoyl-l-glutamic acid, a salt thereof, and/or a combination of two or more thereof.

The surfactant system in some preferred embodiments includes sodium cocoyl glycinate, potassium cocoyl glycinate, or a combination thereof. Without being limited to any particular theory, it was surprisingly discovered that formulating the personal care compositions to have particular amounts of sodium cocoyl glycinate and/or potassium cocoyl glycinate can significantly enhance the foaming characteristics of the personal care composition. In certain embodiments, the personal care composition has a total amount of sodium cocoyl glycinate and/or potassium cocoyl glycinate of about 1.6 to about 6 wt. %, based on the total weight of the personal care composition. For instance, the personal care composition may have a total amount of sodium cocoyl glycinate and/or potassium cocoyl glycinate from about 1.6 to about 6 wt. %, about 1.6 to about 5 wt. %, about 1.6 to about 4 wt. %, about 1.6 to about 3 wt. %; from about 2 to about 6 wt. %, about 2 to about 5 wt. %, about 2 to about 4 wt. %, about 2 to about 3 wt. %; from about 2.5 to about 6 wt. %, about 2.5 to about 5 wt. %, about 2.5 to about 4 wt. %; from about 3 to about 6 wt. %, about 3 to about 5 wt. %, about 3 to about 4 wt. %, or any range or subrange thereof, based on the total weight of the personal care composition.

In some embodiments, the personal care composition comprises a total amount of sodium cocoyl glycinate and/or potassium cocoyl glycinate of about 0.1 to about 1.65 wt. %, based on the total weight of the personal care composition. For instance, the personal care composition may have a total amount of sodium cocoyl glycinate and/or potassium cocoyl glycinate from about 0.5 to about 1.65 wt. %, about 0.8 to about 1.65 wt. %, about 1 to about 1.65 wt. %, about 1.1 to about 1.65 wt. %, about 1.2 to about 1.65 wt. %, about 1.3 to about 1.65 wt. %, about 1.4 to about 1.65 wt. %; from about 0.1 to about 1.5 wt. %, about 0.5 to about 1.5 wt. %, about 0.8 to about 1.5 wt. %, about 1 to about 1.5 wt. %, about 1.1 to about 1.5 wt. %, about 1.2 to about 1.5 wt. %, about 1.3 to about 1.5 wt. %; from about 0.1 to about 1.3 wt. %, about 0.5 to about 1.3 wt. %, about 0.8 to about 1.3 wt. %, about 1 to about 1.3 wt. %, about 1.1 to about 1.3 wt. %; from about 0.1 to about 1.2 wt. %, about 0.5 to about 1.2 wt. %, about 0.8 to about 1.2 wt. %, about 1 to about 1.2 wt. %, about 1.1 to about 1.2 wt. %; from about 0.1 to about 1 wt. %, about 0.5 to about 1 wt. %, about 0.8 to about 1 wt. %; from about 0.1 to about 0.7 wt. %, about 0.4 to about 0.7 wt. %, or any range or subrange thereof, based on the total weight of the personal care composition.

The personal care composition may include one or more anionic surfactants selected from anionic surfactants of plant protein, anionic surfactants of silk protein, and combinations of two or more thereof. In at least one preferred embodiment, the personal care composition includes at least one of anionic surfactant selected from an anionic surfactant of plant protein, an anionic surfactant of silk protein, or a combination thereof. Examples of anionic surfactant of plant protein include sodium cocoyl hydrolyzed keratin, sodium cocoyl hydrolyzed rice protein, sodium cocoyl apple amino acid, sodium cocoyl hydrolyzed soy protein, sodium cocoyl hydrolyzed sweet almond protein, sodium cocoyl hydrolyzed wheat protein, sodium cocoyl hydrolyzed wheat protein glutamate, sodium cocoyl oat amino acids, sodium cocoyl/palmoyl/sunfloweroyl glutamate, potassium lauroyl wheat amino acid, sodium lauroyl wheat amino acid, sodium lauroyl oat amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, and disodium cocoyl glutamate. For instance, the anionic surfactant of plant protein may be selected from potassium lauroyl wheat amino acid, sodium lauroyl wheat amino acid, sodium lauroyl oat amino acid, sodium cocoyl apple amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof. In some embodiments, the anionic surfactant of plant protein is selected from sodium cocoyl hydrolyzed keratin, sodium cocoyl hydrolyzed rice protein, sodium cocoyl apple amino acid, sodium cocoyl hydrolyzed soy protein, sodium cocoyl hydrolyzed sweet almond protein, sodium cocoyl hydrolyzed wheat protein, sodium cocoyl hydrolyzed wheat protein glutamate, sodium cocoyl oat amino acids, sodium cocoyl/palmoyl/sunfloweroyl glutamate, and a combination of two or more thereof. In at least one embodiment, the personal care composition comprises hydrolyzed rice protein, cocoyl apple amino acid, or a combination of two or more thereof.

The personal care composition may include an anionic surfactant of silk protein. For instance, the personal care composition may include sodium cocoyl hydrolyzed silk in certain embodiments.

Additional anionic surfactants that may be incorporated into or, in some cases, excluded from the personal care compositions include sulfate surfactants (e.g., sodium lauryl sulfate, sodium lauryl ether sulfate, etc.), sulfonate surfactants, taurate surfactants, isethionate surfactants, sarcosinate surfactants, sulfonate surfactants, sulfosuccinate surfactants, sulfoacetate surfactants, alkoxylated monoacid surfactants, salts thereof, and combinations of two or more thereof. The anionic surfactant(s) may have an alkyl and/or acyl group comprising 6 to 26 carbon atoms, 8 to 26 carbon atoms, 8 to 21 carbons, 8 to 16 carbon atoms, 10 to 16 carbon atoms, or 10 to 13 carbon atoms. In some instances, the anionic surfactant comprises an alkyl and/or acyl group having more than 26 carbons.

The anionic surfactant may comprise or being selected from sulfonate surfactants, such as those in accordance with the following structure:

R¹—CH═CH—CH₂—SO₃⁻M⁺

in which R¹ is selected from linear or branched alkyl groups having from 9 to 16 carbon, e.g., 11 to 13 carbons, and mixtures thereof; and M is a solubilizing cation. R¹ may be a 14 or 16 carbon linear alkyl group. M may be a cation selected from alkali metal cations (such as sodium or potassium), ammonium cations and substituted ammonium cations (such as alkylammonium, alkanolammonium or glucammonium).

The sulfonate surfactants may comprise sodium, ammonium or potassium salts of sulfonates and/or sodium, ammonium or potassium salts of ether sulfonates. Non-limiting examples of sulfonates can include alpha olefin sulfonates, linear alkylbenzene sulfonates, sodium laurylglucosides hydroxypropylsulfonate and combinations thereof. Additional examples of sulfonate surfactants include olefin sulfonate surfactants, such as sodium C₁₄-C₁₆ olefin sulfonate surfactant and potassium C₁₄-C₁₆ olefin sulfonate surfactants, sodium tridecyl benzene sulfonate, sodium dodecyl benzene sulfonate, or a combination thereof.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or a combination of two or more thereof.

Non-limiting examples of sarcosinates can be selected from the group consisting of sodium lauroyl sarcosinate, sodium cocoyl sarcosinate, sodium myristoyl sarcosinate, TEA-cocoyl sarcosinate, ammonium cocoyl sarcosinate, ammonium lauroyl sarcosinate, dimer dilinoleyl bis-lauroylglutamate/lauroylsarcosinate, disodium lauroamphodiacetate lauroyl sarcosinate, isopropyl lauroyl sarcosinate, potassium cocoyl sarcosinate, potassium lauroyl sarcosinate, sodium cocoyl sarcosinate, sodium lauroyl sarcosinate, sodium myristoyl sarcosinate, sodium oleoyl sarcosinate, sodium palmitoyl sarcosinate, TEA-cocoyl sarcosinate, TEA-lauroyl sarcosinate, TEA-oleoyl sarcosinate, TEA-palm kernel sarcosinate, and combinations thereof.

Non-limiting examples of isethionate surfactants include sodium isethionate, sodium cocoyl isethionate, sodium lauroyl methyl isethionate, and sodium cocoyl methyl isethionate. Sulfonated monoglycerides of fatty acids include sodium coconut monoglyceride sulfonates and the like. Examples of sarcosinate surfactants include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, ammonium lauroyl sarcosinate, and combinations of two or more thereof. Non-limiting examples of alkyl taurate salts include sodium cocoyl taurate, sodium methyl cocoyl taurate, oleoyl taurate. In at least one preferred embodiment, the personal care composition includes one or more anionic surfactant comprising oleoyl taurate and/or a salt thereof.

Isethionate surfactants can also include the reaction product of fatty acids esterified with isethionic acid and neutralized with sodium hydroxide. Fatty acids for isethionate surfactants can be derived from coconut oil or palm kernel oil including amides of methyl tauride. Non-limiting examples of isethionates can be selected from the group consisting of sodium lauroyl methyl isethionate, sodium cocoyl isethionate, ammonium cocoyl isethionate, sodium hydrogenated cocoyl methyl isethionate, sodium lauroyl isethionate, sodium cocoyl methyl isethionate, sodium myristoyl isethionate, sodium oleoyl isethionate, sodium oleyl methyl isethionate, sodium palm kerneloyl isethionate, sodium stearoyl methyl isethionate, or mixtures thereof.

Non-limiting examples of alkyl sulfacetates includes, e.g., alkyl sulfoacetates such as C₄-C₁₈ fatty alcohol sulfoacetates and/or salts thereof. A preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

The anionic surfactant may be selected from taurate surfactants having a structure according to the following formula:

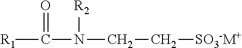

wherein R₁ is a saturated or unsaturated, straight or branched alkyl chain with 6 to 18 carbon atoms, R₂ is H or methyl, and M⁺ is H, sodium, or potassium (e.g., sodium methyl cocoyl taurate).

Additional examples of taurate surfactants include sodium cocoyl taurate, potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations of two or more thereof. In some embodiments, the personal care composition comprises sodium lauroyl methyl taurate (or sodium methyl lauroyl taurate), sodium methyl cocoyl taurate (SMCT), or a combination thereof. In at least one preferred embodiment, the surfactant system comprises sodium methyl cocoyl taurate.

The one or more surfactant(s) may include a nonionic surfactant. The nonionic surfactant(s) may be present in the personal care composition in an amount from about 1 to about 20 wt. %, based on the total weight of the personal care composition. For example, the total amount of nonionic surfactant(s) in the personal care composition may be from about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 7 wt. %, about 1 to about 6 wt. %, about 1 to about 5 wt. %; from about 3 to about 20 wt. %, about 3 to about 18 wt. %, about 3 to about 16 wt. %, about 3 to about 14 wt. %, about 3 to about 12 wt. %, about 3 to about 10 wt. %, about 3 to about 8 wt. %, about 3 to about 7 wt. %, about 3 to about 6 wt. %, about 3 to about 5 wt. %; from about 5 to about 20 wt. %, about 5 to about 18 wt. %, about 5 to about 16 wt. %, about 5 to about 14 wt. %, about 5 to about 12 wt. %, about 5 to about 10 wt. %, about 5 to about 8 wt. %; from about 7 to about 20 wt. %, about 7 to about 18 wt. %, about 7 to about 16 wt. %, about 7 to about 14 wt. %, about 7 to about 12 wt. %, about 7 to about 10 wt. %; from about 9 to about 20 wt. %, about 9 to about 18 wt. %, about 9 to about 16 wt. %, about 9 to about 14 wt. %, about 9 to about 12 wt. %; from about 11 to about 20 wt. %, about 11 to about 18 wt. %, about 11 to about 16 wt. %, about 11 to about 13 wt. %; from about 13 to about 20 wt. %, or about 13 to about 18 wt. %, including ranges and subranges thereof, based on the total weight of the personal care composition.

The nonionic surfactant may be a glucoside surfactant, a lactylate surfactant, alkylamides, alkanolamides, polyoxyalkylenated nonionic surfactants, polyglycerolated nonionic surfactants, or ethoxylated fatty esters. Examples of glucoside surfactants include alkyl glucoside, such as C₆₋₂₅ alkyl glucoside, C₈₋₁₈ alkyl glucoside, C₁₀₋₁₈ alkyl glucoside or C₁₀₋₁₆ alkyl glucoside. For example, the personal care composition may include glucoside surfactants selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination thereof.

The lactylate surfactant, when present in the personal care composition, may have a carbon chain between C₈ and C₁₆. Non-limiting examples of lactylate surfactant include decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, and a combination of two or more thereof.

Examples alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one —OH group).

The nonionic surfactants may be selected from ethoxylated fatty esters, such as adducts of ethylene oxide with esters of lauric acid, palmitic acid, stearic acid, behenic acid, and mixtures thereof, including those containing from 9 to 100 oxyethylene groups. For example, the ethoxylated fatty esters comprise PEG-9 laurate to PEG-50 laurate; PEG-9 palmitate to PEG-50 palmitate; PEG-9 stearate to PEG-50 stearate; PEG-9 palmitostearate to PEG-50 palmitostearate; PEG-9 behenate to PEG-50 behenate; polyethylene glycol 100 EO monostearate (such as, PEG-100 stearate); and combinations of two or more thereof. As examples of glyceryl esters of fatty acids, glyceryl stearate (glyceryl mono-, di- and/or tristearate), glyceryl ricinoleate, and a combination of two or more thereof may be incorporated in the personal care composition. Examples of glyceryl esters of C₈-C₂₄ alkoxylated fatty acids include polyethoxylated glyceryl stearate (glyceryl mono-, di- and/or tristearate), such as PEG-20 glyceryl stearate.

Additionally or alternatively, the surfactants may include one or more amphoteric surfactants. Amphoteric surfactants are typically characterized by a combination of high surfactant activity, lather forming and mildness. The amphoteric surfactant may comprise a substituent containing 8 to 18 carbon atoms and a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched and wherein one of the aliphatic substituents contains about 8 to about 18 carbon atoms and one contains an anionic water solubilizing group, e.g., carboxy, sulfonate, sulfate, phosphate, or phosphonate.

The amphoteric surfactants may include alkyl amphoproprionates, betaines, alkyl sultaines, alkyl amphoacetates, and mixtures thereof. In some instances, the personal care composition includes one or more alkyl amphopropionates. The personal care composition may include an amphoteric surfactant selected from betaine surfactants (also referred to as betaines). Examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine, sulfobetaines such as coco dimethyl sulfopropyl betaine, stearyl dimethyl sulfopropyl betaine, amido betaines, amidosulfobetaines, and the like. Preferably, the betaine surfactant is selected from coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and combinations of two or more thereof. In some embodiments, at least one betaine surfactant is selected from coco betaine, cocamidopropyl betaine, behenyl betaine, capryl/capramidopropyl betaine, and lauryl betaine, and a combination of two or more thereof. In further embodiments, the betaine surfactant is selected from cocamidopropyl betaine, lauryl dimethyl betaine, cetyl dimethyl betaine, cocoamphodiacetate, and a combination of two or more thereof. In at least one embodiment, the personal care composition comprises coco betaine, cocamidopropyl betaine, or a combination thereof.

The amphoteric surfactant may, in some cases, be selected from sultaines, also known as sulfobetaines, and hydroxy sultaines, e.g., cocamidopropyl hydroxysultain. The sultaines may be an alkyl sultaines having from 6-20 carbons, 8-18 carbons, 8-16 carbons, or 10-16 carbons. The amphoteric surfactant may comprise or be selected from hydroxysultaine surfactants, such as cocamidopropyl hydroxysultaine surfactants.

The surfactant system may, in certain embodiments, comprise two or more acyl amino acid surfactants, wherein at least one acyl amino acid surfactant is selected from sodium cocoyl glycinate and/or potassium cocoyl glycinate. For example, the surfactant system may comprise or consist of a total amount of sodium cocoyl glycinate and/or potassium cocoyl glycinate of about 0.1 to about 1.65 wt. % and at least one of an acyl amino acid surfactant other than sodium cocoyl glycinate or potassium cocoyl glycinate, such as an acyl amino acid surfactant described above. The surfactant system may comprise or consist of a total amount of sodium cocoyl glycinate and/or potassium cocoyl glycinate of about 0.1 to about 1.65 wt. % and at least one of an acyl amino acid surfactant other than sodium cocoyl glycinate or potassium cocoyl glycinate, a sulfonate surfactant (e.g. an alkali metal olefin sulfonate surfactant), an anionic taurate surfactant, an anionic glutamate surfactant, an anionic isethionate surfactant, a betaine surfactant, a glucoside surfactant, or a combination of two or more thereof.

The personal care composition may include one or more polyol(s). The polyol(s) may be present in an amount from about 0.5 to about 10 wt. %, based on the total weight of the personal care composition. For example, the amount of polyol(s) in the personal care composition may be from about 0.5 to about 8 wt. %, about 0.5 to about 6 wt. %, about 0.5 to about 5 wt. %, about 0.5 to about 4 wt. %, about 0.5 to about 3 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %; from about 2 to about 10 wt. %, about 2 to about 8 wt. %, about 2 to about 6 wt. %, about 2 to about 5 wt. %, about 2 to about 4 wt. %, about 2 to about 3 wt. %; from about 3 to about 10 wt. %, about 3 to about 8 wt. %, about 3 to about 6 wt. %, about 3 to about 5 wt. %; from about 5 to about 10 wt. %, about 5 to about 8 wt. %; from about 7 to about 10%, about 8 to about 10 wt. %, or any range or subrange thereof, based on the total weight of the personal care composition.

The polyol(s) may be chosen from glycols or compounds with numerous hydroxyl groups. In some cases, the polyol(s) are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms. The polyol(s) may be a humectant.

Polyols that may be included in the personal care composition, in certain instances, include ethylene glycol, propylene glycol, butylene glycol, hexylene glycol, glycerin, diglycerin, diethylene glycol, dipropylene glycol, and mixtures thereof. In some cases, the personal care composition comprises glycerin, and optionally one or more polyols other than glycerin. Non-limiting examples of polyols that may, optionally, be included in the personal care include alkanediols such as 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, or a combination of two or more thereof. In at least one embodiment, the personal care composition includes one or more polyol(s) comprising panthenol, pantothenic acid, or a combination thereof. Additional examples of glycol ethers include, e.g., monomethyl, monoethyl and monobutyl ethers of ethylene glycol, propylene glycol of ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol.

In some cases, the one or more polyols may include ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, or a combination of two or more thereof. In certain embodiments, the personal care composition may include polyol(s) selected from glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600.

Additionally or alternatively, the personal care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof.

The personal care composition may include one or more humectant, e.g., in an amount from about 0.1 to about 4 wt. %, based on the total weight of the personal care composition. In some embodiments, the one or more humectant is present in the personal care composition in amount from about 0.1 to about 4 wt. %, about 0.1 to about 3 wt. %, about 0.1 to about 2 wt. %, about 0.1 to about 1 wt. %; from about 0.1 to about 4 wt. %, about 0.1 to about 3 wt. %, about 0.1 to about 2 wt. %, about 0.1 to about 1 wt. %; from about 0.1 to about 4 wt. %, about 0.1 to about 3 wt. %, about 0.1 to about 2 wt. %, about 0.1 to about 1 wt. %; from about 0.2 to about 4 wt. %, about 0.2 to about 3 wt. %, about 0.2 to about 2 wt. %, about 0.2 to about 1 wt. %; from about 0.5 to about 4 wt. %, about 0.5 to about 3 wt. %, about 0.5 to about 2 wt. %, about 0.5 to about 1 wt. %; from about 0.7 to about 4 wt. %, about 0.7 to about 3 wt. %, about 0.7 to about 2 wt. %, about 0.7 to about 1 wt. %; from about 1 to about 4 wt. %, about 1 to about 3 wt. %, about 1 to about 2 wt. %; from about 1.5 to about 4 wt. %, about 1.5 to about 3 wt. %, about 1.5 to about 2 wt. %; from about 2 to about 4 wt. %, about 2 to about 3 wt. %; from about 2.5 to about 4 wt. %, about 2.5 to about 3 wt. %, about 3 to about 4 wt. %, or any range or subrange thereof, based on the total weight of the personal care composition.

The humectants may be selected from humectants other than polyols. For instance, the humectants may comprise or consist of those humectants other than polyols, such as pyrrolidone carboxylic acid (PCA), pyroglutamate acid, pyrrolidone carboxylic acid glyceryl oleate (PCA glyceryl oleate), a salt thereof, and a combination of two or more thereof. Examples of salts of pyrrolidone carboxylic acid include sodium pyrrolidone carboxylate, zinc pyrrolidone carboxylate, and potassium pyrrolidone carboxylate. Examples of salts of pyroglutamate acid include sodium L-pyroglutamat and sodium D-pyroglutamate. The humectants may be selected from polyols such as glycerin, saccharide isomerate butylene glycol, 1,3-propanediol, sorbitol, xylitol or low molecular weight polyethylene glycols (PEGs), polyoxyethylenes; or a combination of two or more thereof. In some preferred embodiments, the humectant comprises pyrrolidone carboxylic acid (PCA) and/or a salt thereof (e.g., sodium PCA).

The personal care composition may optionally include one or more thickening agent(s). The amount of thickening agent(s) in the personal care composition, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

The thickener(s) may be chosen from carboxylic acid polymers; crosslinked polyacrylate polymers; polyacrylamide polymers; gums; polysaccharides; and combinations of two or more thereof. Examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof.

Non-limiting examples of carboxylic acid polymers include crosslinked carboxylic acid polymers containing one or more monomers derived from acrylic acid, substituted acrylic acids, and salts and esters of these acrylic acids and the substituted acrylic acids, such as carbomers. Carbomers are long polymeric chains of acrylic acid. An exemplary structure of a monomer of a carbomer is shown below.

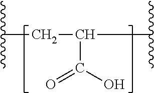

The carbomer may be selected from Carbopols. For instance, the personal care composition may include, or in some cases exclude, a carbomer selected from Carbopol 2984, Carbopol 934, Carbopol Ultrez 10, Carbopol 940, Carbopol 941, Carbopol 956 Carbopol 981, Carbopol 2020, Carbopol Aqua SF-1, and a combination of two or more thereof. In certain preferred embodiments, the carbomer is C₁₀₋₃₀ alkyl acrylate cross-polymer.

Non-limiting examples of gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and a combination of two or more thereof.

In certain cases, the personal care composition may be formulated to have a thickening agent selected from thickening polymers, such as polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (e.g., carboxymethyl cellulose, methyl cellulose, methylhydroxypropyl cellulose, hydroxyethyl cellulose, hydroxypropyl cellulose, cellulose sulfate sodium salt), and/or starch derivatives (e.g., starch oxide, dialdehyde starch, dextrin, British gum, acetyl starch, starch phosphate, carboxymethyl starch, hydroxyethyl starch, hydroxypropyl starch). Additionally, the thickening agents may include polymeric thickeners selected from the group consisting of ammonium polyacryloyldimethyl taurate, ammonium acryloyldimethyltaurate/VP copolymer, acrylates copolymers, polyacrylamide, carbomer, acrylates/C10-30 alkyl acrylate crosspolymer, and a combination of two or more thereof.

The thickening agents may be chosen from xanthan gum, guar gum, biosaccharide gum, cellulose, acacia seneca gum, sclerotium gum, agarose, pectin, gellan gum, hyaluronic acid, and a combination of two or more thereof. Additionally, the thickening agents may include polymeric thickeners selected from the group consisting of ammonium polyacryloyldimethyl taurate, ammonium acryloyldimethyltaurate/VP copolymer, acrylates copolymers, polyacrylamide, carbomer, acrylates/C10-30 alkyl acrylate crosspolymer, and a combination of two or more thereof.

In some embodiments, the thickening agent is selected from among hydroxypropyl methylcellulose, polyethylene glycols (PEGs), polyacrylic acids, cross-linked homopolymer of acrylic acid, and acrylates of C₁₀₋₃₀ alkyl acrylate crosspolymer. The thickening agents may be chosen from xanthan gum, guar gum, biosaccharide gum, cellulose, acacia seneca gum, sclerotium gum, agarose, pectin, gellan gum, hyaluronic acid, and a combination of two or more thereof.

Additionally or alternatively, the personal care composition may include one or more skin soothing agent(s), such as bisabolol, aloe vera extract, allantoin, licorice extract, glycyrrhizin, or combinations thereof. In some preferable embodiments, the personal care compositions includes two or more skin soothing agents. For example, the personal care composition may include two, three, four, five, six, seven, or any range thereof, of skin soothing agents.

In some embodiments, the soothing agents may be selected from natural extracts comprising: bisabolol, Centella asiatica extract, Polygonum cuspidatum root extract, Scutellaria baicalensis root extract, Camellia sinensis leaf extract, Glycyrrhiza glabra (licorice) root extract, Chamomilla recutita (matricaria) flower extract, Rosmarinus officinalis (rosemary) leaf extract, tea (Camellia sinensis) leaf extract, Poria cocos extract, Phragmites karka extract, lidocaine, chia seed extract, Rosa centifolia flower extract, aloe vera, allantoin, D-panthenol, turmeric, avocado oil, and lichen extract, or a combination of two or more thereof.

The personal care composition may include the soothing agent(s) in an amount from about 0.05 to about 5 wt. %, based on the total weight of the personal care composition. In some embodiments, the total amount of soothing agent(s) in the personal care composition is from about 0.05 to about 5 wt. %, about 0.05 to about 4 wt. %, about 0.05 to about 3 wt. %, about 0.05 to about 2 wt. %, about 0.05 to about 1 wt. %, about 0.05 to about 0.7 wt. %; from about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.7 wt. %; from about 0.3 to about 5 wt. %, about 0.3 to about 4 wt. %, about 0.3 to about 3 wt. %, about 0.1 to about 2 wt. %, about 0.3 to about 1 wt. %, about 0.3 to about 0.7 wt. %; from about 0.5 to about 5 wt. %, about 0.5 to about 4 wt. %, about 0.5 to about 3 wt. %, about 0.5 to about 2 wt. %, about 0.5 to about 1 wt. %, about 0.5 to about 0.7 wt. %; from about 1 to about 5 wt. %, about 1 to about 4 wt. %, about 1 to about 3 wt. %, about 1 to about 2 wt. %, about 1 to about 1.5 wt. %; from about 1.5 to about 5 wt. %, about 1.5 to about 4 wt. %, about 1.5 to about 3 wt. %, about 1.5 to about 2 wt. %; from about 2 to about 5 wt. %, about 2 to about 4 wt. %, about 2 to about 3 wt. %; from about 3 to about 5 wt. %, about 3 to about 4 wt. %, or any range or subrange thereof, based on the total weight of the personal care composition.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

In certain embodiments, the personal care composition has a pH of from about 3.5 to about 10. In other embodiments, the personal care composition has a pH of from about 3.5 to about 8. In some embodiments, the personal care composition has a pH of from about 3.5 to about 7.5. In other embodiments, the personal care composition has a pH of from about 3.5 to about 7. In further embodiments, the personal care composition has a pH of from about 3.5 to about 6.5. Still other embodiments provide personal care compositions having a pH of from about 3.5 to about 6. While other embodiments provide personal care compositions having a pH of from about 3.5 to about 5.5. Yet other embodiments provide personal care compositions having a pH of from about 3.5 to about 5. In certain embodiments, the personal care composition has a pH of from about 3.75 to about 4.5. In certain embodiments, the personal care composition is a body wash, having a pH of from about 4 to about 6.

Additional ingredients may be present in the personal care composition. These include ingredients to thicken, preserve, emulsify, add fragrance, adjust the pH, and color. The personal care compositions may include any of the following additional ingredients in an amount of from about 0.01 to about 5 wt. %. In some instances, the amount of additional ingredients present in the personal care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the personal care composition.

The personal care composition can comprise additional ingredients, such as proteins (e.g., hydrolyzed vegetable protein, hydrolyzed wheat protein, hydrolyzed milk protein, hydrolyzed silk and hydrolyzed collagen), emollients (e.g., esters, isopropyl myristate, decyl oleate, C₁₂₋₁₅ alkyl benzoate), botanicals (e.g., chamomile, aloe, rosemary), as well as preservatives, dyes, pH adjusters and chelating agents. Additional examples of vitamins that may be included in some personal care compositions include tocopherol, retinol, and ascorbic acid. The additional ingredients may include clays, such as magnesium silicate and/or magnesium aluminum silicate.

Non-limiting examples of fragrances and perfumes include odor compounds selected from: 7-acetyl-1,2,3,4,5,6,7,8-octahydro-1,1,6,7-tetramethylnaphthalene, α-ionone, β-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl)propionaldehyde, ethylvanillin, heliotropin, hexylcinnamaldehyde, amylcinnamaldehyde, 2-methyl-2-(isopropylphenyl)propionaldehyde, coumarin, γ-decalactone, cyclopentadecanolide, 16-hydroxy-9-hexadecenoic acid lactone, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho[2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, and tert-butylcyclohexyl acetate.

Other fragrances may include odor compounds selected from essential oils, resinoids and resins from a large number of sources, such as, for example, Peru balsam, olibanum resinoid, styrax, labdanum resin, nutmeg, cassia oil, benzoin resin, coriander, and lavandin.

Further suitable fragrances include odor compounds selected from phenylethyl alcohol, terpineol, linalool, linalyl acetate, geraniol, nerol, 2-(1,1-dimethylethyl)cyclo-hexanol acetate, benzyl acetate, and eugenol. The fragrances or perfumes can be used as single substances or in a mixture with one another.

The personal care composition may further comprise one or more colorants. Exemplary colorants can include natural or uncertified colors from natural sources or certified colors for the effect of color. In some embodiments, the colorant can include dyes, certified aluminum lakes, or colors derived from a natural source. The colorant may be water-based, oil-based, or dry. The colorants can be primary colors, blends of colors or discrete mixtures of colors, such as confetti. Some color agents (colorants) are known as FD&C dyes.

The concentrations of the colorant in the personal care composition may be from trace amount to about 0.6 wt. %, based on the total weight of the personal care composition, or any of the amount disclosed above for the additional ingredients. The concentrations of the coloring agent(s) in the personal care composition may be from trace amount to about 0.6 wt. %, e.g., from about 0.001 to about 0.5 wt. %, about 0.002 to about 0.4 wt. %, about 0.005 to about 0.4 wt. %, about 0.015 to about 0.35 wt. %, about 0.025 to about 0.35 wt. %, about 0.05 to about 0.35 wt. %; from about 0.001 to about 0.2 wt. %, about 0.002 to about 0.2 wt. %, about 0.005 to about 0.2 wt. %, about 0.015 to about 0.2 wt. %, about 0.025 to about 0.2 wt. %, about 0.05 to about 0.2 wt. %; from about 0.001 to about 0.1 wt. %, about 0.002 to about 0.1 wt. %, about 0.005 to about 0.1 wt. %, about 0.015 to about 0.1 wt. %, about 0.025 to about 0.1 wt. %, about 0.05 to about 0.1 wt. %, or any range or subrange thereof, based on the total weight of the personal care composition.

The personal care composition may include particulates, which may be opacifiers. Non-limiting examples of particulates and/or opacifiers include, without limitation, magnesium silicate, fumed silica, calcined clay, rice starch, silica and/or hydrated silica, titanium dioxide particles, zinc oxide particles, aluminum oxide particles, tin oxide particles, calcium oxide particles, magnesium oxide particles, barium oxide particles, silica particles, zirconium silicate particles, mica particles, talc particles, tetracalcium phosphate particles, amorphous calcium phosphate particles, alpha-tricalcium phosphate particles, beta-tricalcium phosphate particles, hydroxyapatite particles, calcium carbonate particles, zinc phosphate particles, silicon dioxide particles, zirconium silicate particles, or the like, or mixtures and combinations thereof. In some embodiments, the personal care composition comprises one or more particulate(s) and/or opacifier(s) selected from the group consisting of fumed silica, calcined clay, rice starch, silica (e.g., hydrated silica), and a combination of two or more thereof.

The amount of the above listed particulates(s) and/or opacifier(s) present in the personal care composition may be from trace amount to about 0.6 wt. %, based on the total weight of the personal care composition, or any of the amount disclosed above for the additional ingredients. In some cases, the amount of the above listed particulates(s) and/or opacifier(s) present in the personal care composition is from about 0.001 to about 0.5 wt. %, about 0.002 to about 0.4 wt. %, about 0.005 to about 0.4 wt. %, about 0.015 to about 0.35 wt. %, about 0.025 to about 0.35 wt. %, about 0.05 to about 0.35 wt. %; from about 0.001 to about 0.2 wt. %, about 0.002 to about 0.2 wt. %, about 0.005 to about 0.2 wt. %, about 0.015 to about 0.2 wt. %, about 0.025 to about 0.2 wt. %, about 0.05 to about 0.2 wt. %; from about 0.001 to about 0.1 wt. %, about 0.002 to about 0.1 wt. %, about 0.005 to about 0.1 wt. %, about 0.015 to about 0.1 wt. %, about 0.025 to about 0.1 wt. %, about 0.05 to about 0.1 wt. %, or any range or subrange thereof, based on the total weight of the personal care composition.

In certain embodiments, in order to prevent ingredients from separating, emulsifiers may be added. The amount of additional emulsifiers present in the personal care composition may be any of the amounts (or ranges thereof) recited for the additional ingredients.

In other embodiments, the personal composition may include any of following materials in any desired amount to achieve a desired effect in the composition (amounts that can be used in some embodiments are provided): one or more alkaline salts, for example, sodium chloride, sodium sulfate, sodium carbonate, sodium bicarbonate and/or their equivalents (0 to 5% by weight); sequestrants, for example, tetrasodium EDTA, and/or their equivalents (0 to 2% by weight); biocides, for example, Triclosan (2,4,4′-trichloro-2′-hydroxydiphenyl ether), DMDM hydantoin, formaldehyde and/or imidazolidinyl urea, and/or their equivalents (0 to 2% by weight); organic acids, for example, citric acid and/or formic acid and/or their equivalents (0 to 2% by weight); viscosity modifiers (0 to 2% by weight); fragrances and/or perfumes (0 to 5% by weight); preservatives, for example, sodium metabisulfite, phenoxyethanol, formaldehyde solution, parabens, pentanediol or sorbic acid (0 to 2% by weight); and pearlizing agents, for example, glycol distearic esters, such as ethylene glycol distearate, but also fatty acid monoglycol esters (0 to 3% by weight); stabilizers, for example, metal salts of fatty acids, such as e.g. magnesium stearate, aluminum stearate and/or zinc stearate (0 to 2% by a weight).

The personal care compositions typically include an amount of water. The amount of water present in the personal care composition may be from about 10 to about 90 wt. %, about 15 to about 90 wt. %, about 20 to about 90 wt. %, about 30 to about 90 wt. %, about 40 to about 90 wt. %, about 50 to about 90 wt. %, about 60 to about 90 wt. %, about 70 to about 90 wt. %, about 80 to about 90 wt. %; about 10 to about 80 wt. %, about 15 to about 80 wt. %, about 20 to about 80 wt. %, about 30 to about 80 wt. %, about 40 to about 80 wt. %, about 50 to about 80 wt. %, about 60 to about 80 wt. %, about 70 to about 80 wt. %; about 10 to about 70 wt. %, about 15 to about 70 wt. %, about 20 to about 70 wt. %, about 30 to about 70 wt. %, about 40 to about 70 wt. %, about 50 to about 70 wt. %, about 60 to about 70 wt. %; about 10 to about 60 wt. %, about 15 to about 60 wt. %, about 20 to about 60 wt. %, about 30 to about 60 wt. %, about 40 to about 60 wt. %, about 50 to about 60 wt. %; about 10 to about 50 wt. %, about 15 to about 50 wt. %, about 20 to about 50 wt. %, about 30 to about 50 wt. %, about 40 to about 50 wt. %, including any ranges and subranges thereof, based on the total weight of the personal care composition.

The personal care composition may be contained in or provided in any suitable container such as an aerosol can, tube or container with a porous cap, roll-on container, bottle, container with an open end, etc. One of ordinary skill in the art would be able to identify containers for containing the personal care compositions disclosed herein. Disclosures of containers can be found in U.S. Pat. Nos. 9,173,530, 5,289,950, and PCT Publication No. WO 2007/144793 A, which are incorporated herein by reference in their entirety for all purposes.

### EXAMPLES

### Example 1

A non-limiting, exemplary self-foaming personal care composition (Example Composition A) was prepared in accordance with an aspect of the invention. The formulation for Example Composition A is provided in Table 1.

TABLE 1

| US INCI Name | Ex. A (wt. %) |
|---|---|
| Sodium Cocoyl Glycinate | 6 |
| Sodium Cocoyl Apple Amino Acids | 2.2 |
| Isopentane | 1.5 |
| Octyl Isononanoate | 0.75 |
| Isononyl Isononanoate | 1 |
| Ethylhexyl Isononanoate | 0.2 |
| Phenoxyethanol, ethylhexylglycerin, | 1 |
| Phenyl Propyl Alcohol, Caprylyl Glycol, | |
| 1,3-propanediol, and DL-Alpha- | |
| Tocopherol | |
| Polyhydroxystearic acid | 0.5 |
| Cocamidopropyl PG-Dimonium Chloride | 0.2 |
| Phosphate | |
| Glycerin | 2 |
| Magnesium Silicate | 1.1 |
| Sodium Bisulfite | 0.01 |
| Niacinamide | 0.2 |
| Licorice Root Extract | 0.025 |
| Sodium PCA | 0.2 |
| Acrylates Copolymer | 3.8 |
| Sodium hydroxide | 0.3 |
| Fragrance | 0.03 |
| Water | Q.S. to 100 |

### Example 2

Compositions containing pentane, one or more fatty acid, and one or more fatty ester were evaluated to determine their respective boiling points. The compositions were prepared in accordance with the formulas presented in Table 2 along with the boiling points of the respective compositions.

TABLE 2

| | Boil Point (BP) | % BP |
|---|---|---|
| Composition | (° C.) | Increase* |
| Pentane | 36.1 | 0 |
| Pentane and Octanoic Acid in a ratio of Pentane | 41.9 | 16 |
| to Octanoic of 1:1 (w/w) | | |
| Pentane, Stearic Acid, and Isopropyl Myristate in | 45.4 | 26 |
| a weight ratio of Pentane to Stearic Acid to | | |
| Isopropyl Myristate of 1:1:1 (w/w) | | |
| Pentane, Euxyl 9010, and Octyl Isononanoate in a | 44.9 | 24 |
| weight ratio of Pentane to Euxyl 9010 to Octyl | | |
| Isononanoate of 1:1:1 (w/w/w) | | |
| Pentane and Octyl Isononanoate in a weight ratio | 42.7 | 18 |
| of Pentane to Octyl Isononanoate of 1:1 (w/w) | | |
| Pentane and Polyhydroxystearic Acid in a weight | 38.1 | 6 |
| ratio of Pentane to Polyhydroxystearic Acid of 1:1 | | |
| (w/w) | | |
| Pentane and Isononyl Isononanoate in a weight | 42.7 | 18 |
| ratio of Pentane to Isononyl Isononanoate of 1:1 | | |
| (w/w) | | |
| Pentane, Octyl Isononanoate, and Isononyl | 49.4 | 37 |
| Isononanoate in a weight ratio of Pentane to Octyl | | |
| Isononanoate to Isononyl Isononanoate of 1:1:1 | | |
| (w/w/w) | | |
| Pentane and Coconut Fatty Acids in a ratio of | 42.3 | 17 |
| Pentane to Octanoic of 1:1 (w/wv) | | |

*Calculated by dividing the boiling point of mixture by the boiling point of pentane (36.1° C.)

It is believed that an increase in the boiling point may contribute to an increased processing stability as well as increased stability during storage even in unpressurized cosmetic packaging.

### Example 3

Three non-limiting exemplary personal care compositions (Example Compositions B-D) and two comparative personal care compositions (Comparative Compositions 1 and 2) were prepared in accordance with aspects of the invention. The formulation for Example Compositions B-D and Comparative Compositions 1 and 2 are shown in Table 3.

Example Compositions B-D and Comparative Compositions 1 and 2 were assessed to determine the amount of foam yielded from each composition after 4 hours. Specifically, samples weighing 60 grams for each of Example Compositions B-D and Comparative Compositions 1 and 2 were added to jars, followed by hand-mixing with a spatula for 50 seconds. The samples were then left uncapped and monitored for at least 4 hours to elucidate any differences in the generation of self-foam after the addition of the isopentane premixes. The amount of foam produced while the sample was in an open jar in ambient conditions was measured with a ruler (from the starting point of the liquid to the top of the self-generated foam layer) and reported in Table 4.

TABLE 3

| | Base | | | | | |
|---|---|---|---|---|---|---|
| US INCI Compound | (5% Hole) | Comp. 1 | Comp. 2 | Ex. B | Ex. C | Ex. D |
| Name | (wt. %) | (wt. %) | (wt. %) | (wt. %) | (wt. %) | (wt. %) |
| Sodium Cocoyl Glycinate | 4 | 4 | 4 | 4 | 4 | 4 |
| Sodium Cocoyl Apple | 3 | 3 | 3 | 3 | 3 | 3 |
| Amino Acids | | | | | | |
| Glycerin and Hexylene | 2.2 | 2.2 | 2.2 | 2.2 | 2.2 | 2.2 |
| Glycol | | | | | | |
| Magnesium Silicate | 0.9-1.5 | 0.9-1.5 | 0.9-1.5 | 0.9-1.5 | 0.9-1.5 | 0.9-1.5 |
| Niacinamide | 0.1-0.4 | 0.1-0.4 | 0.1-0.4 | 0.1-0.4 | 0.1-0.4 | 0.1-0.4 |
| Licorice Root Extract | 0.01-0.05 | 0.01-0.05 | 0.01-0.05 | 0.01-0.05 | 0.01-0.05 | 0.01-0.05 |
| Sodium PCA | 0.1-0.4 | 0.1-0.4 | 0.1-0.4 | 0.1-0.4 | 0.1-0.4 | 0.1-0.4 |
| Acrylates Copolymer | 12.5 | 12.5 | 12.5 | 12.5 | 12.5 | 12.5 |
| NaOH and Sodium | 1.1 | 1.1 | 1.1 | 1.1 | 1.1 | 1.1 |
| Chloride | | | | | | |
| Fragrance | 0.03% | 0.03% | 0.03% | 0.03% | 0.03% | 0.03% |
| Sodium Cocamidopropyl | 0.2 | 0.2 | 0.2 | 0.2 | 0.2 | 0.3 |
| PG-Dimonium Chloride | | | | | | |
| Phosphate | | | | | | |
| Phenylpropanol, Caprylyl | 1.1 | 1.1 | 1.1 | 1.1 | 2.1 | 1.9 |
| Glycol, Propanediol, | | | | | | |
| Tocopherol, | | | | | | |
| Phenoxyethanol, | | | | | | |
| Ethylhexylglycerin, | | | | | | |
| Sodium Bisulfite, and | | | | | | |
| Tetrasodium Glutamate | | | | | | |
| Diacetate | | | | | | |
| Isopentane | | 2 | 2 | 2 | 2 | 2 |
| Coconut Acid | 0.4 | 0.4 | 0.4 | 0.4 | 0.4 | 0.4 |
| Polyhydroxystearic Acid | 0.5 | 0.5 | 0.5 | 0.5 | 0.5 | 0.7 |
| Ethylhexyl Isononanoate | 0.2 | 0.2 | 0.2 | 0.2 | 0.2 | 0.4 |
| Octyl Isononanoate | | | | 1.5 | 1 | 0.8 |
| Isononyl Isononanoate | 0.2 | 0.2 | 0.2 | 1.7 | 1.2 | 1.1 |
| Silicone (0.65 cst) | | | 3 | | | |
| DI Water | | 3 | | | | |
| DI Water | Q.S. | Q.S. | Q.S. | Q.S. | Q.S. | Q.S. |
| | to 95 | to 100 | to 100 | to 100 | to 100 | to 100 |
| Total | 95 | 100 | 100 | 100 | 100 | 100 |

TABLE 4

| | Base | Total | Height |
|---|---|---|---|
| | Height | Height | Change |
| Run | (cm) | (cm) | (%) |
| Comp. 2 | 4.4 | 6.5 | 32% |
| Ex. B | 4.35 | 5.5 | 21% |
| Ex. C | 4.2 | 4.9 | 14% |
| Ex. D | 4.4 | 4.9 | 10% |
| Comp. 1 | 4.2 | 6.3 | 33% |

### Example 4

Eleven non-limiting, exemplary self-foaming personal care compositions (Example Compositions E-P) were prepared in accordance with aspects of the invention. A comparative composition (Comparative Composition 3) was also prepared, which did not have disiloxane, dimethicone, coconut acid, or vitamin E. The formulations for Example Compositions E-P and Comparative Composition 3 are provided in Table 5.

TABLE 5

| | Ex. E | Ex. F | Ex. G | Ex. H | Ex. I | Ex. J |
|---|---|---|---|---|---|---|
| US INCI Name | (wt. %) | (wt. %) | (wt. %) | (wt. %) | (wt. %) | (wt. %) |
| Sodium Cocoyl Glycinate | 3.7-4.5 | 3.7-4.5 | 3.7-4.5 | 3.7-4.5 | 3.7-4.5 | 3.7-4.5 |
| Sodium Cocoyl | 0.7-1 | 0.7-1 | 0.7-1 | 0.7-1 | 0.7-1 | 0.7-1 |
| Hydrolyzed Collagen and | | | | | | |
| Glutamic Acid-N,N- | | | | | | |
| Diacetic Acid | | | | | | |
| Isopentane | 1 | 1 | 1 | 1 | 1 | 1 |
| Disiloxane | 0.5 | 0.75 | 1 | 1.5 | | |
| Dimethicone | | | | | 0.5 | 1 |
| Coconut Acid | 0.4 | 0.4 | 0.4 | 0.4 | 0.4 | 0.4 |
| Tocopheryl Acetate | | | | | | |
| Alpha-Tocopherol | x < | x < | x < | x < | x < | x < |
| | 0.001 | 0.001 | 0.001 | 0.001 | 0.001 | 0.001 |
| Isononyl isononanoate and | 0.5 | 0.5 | 0.5 | 0.5 | 0.5 | 0.5 |
| Ethylhexyl Isononanoate | | | | | | |
| Polyhydroxystearic Acid | 0.5 | 0.5 | 0.5 | 0.5 | 0.5 | 0.5 |
| Cocamidopropyl Pg- | 0.15-3 | 0.15-3 | 0.15-3 | 0.15-3 | 0.15-3 | 0.15-3 |
| Dimonium Chloride | | | | | | |
| Phosphate | | | | | | |
| Phenoxyethanol, and | 1 | 1 | 1 | 1 | 1 | 1 |
| Ethylhexylglycerin | | | | | | |
| Glycerin | 1-3.5 | 1-3.5 | 1-3.5 | 1-3.5 | 1-3.5 | 1-3.5 |
| Hexylene Glycol, 1,3- | 0.2 | 0.2 | 0.2 | 0.2 | 0.2 | 0.2 |
| Propanediol, and Caprylyl | | | | | | |
| Glycol | | | | | | |
| Magnesium Silicate | 1-1.5 | 1-1.5 | 1-1.5 | 1-1.5 | 1-1.5 | 1-1.5 |
| Sodium Bisulfite, | 0.4-0.5 | 0.4-0.5 | 0.4-0.5 | 0.4-0.5 | 0.4-0.5 | 0.4-0.5 |
| Niacinamide, Licorice | | | | | | |
| Root Extract, and Sodium | | | | | | |
| PCA | | | | | | |
| Phenyl Propyl Alcohol | 0.01-0.1 | 0.01-0.1 | 0.01-0.1 | 0.01-0.1 | 0.01-0.1 | 0.01-0.1 |
| Acrylates Copolymer | 12-13 | 12-13 | 12-13 | 12-13 | 12-13 | 12-13 |
| Sodium hydroxide | 0.1-0.5 | 0.1-0.5 | 0.1-0.5 | 0.1-0.5 | 0.1-0.5 | 0.1-0.5 |
| Sodium chloride | 0.8-1.4 | 0.8-1.4 | 0.8-1.4 | 0.8-1.4 | 0.8-1.4 | 0.8-1.4 |
| Fragrance Agents | 0.01-0.1 | 0.01-0.1 | 0.01-0.1 | 0.01-0.1 | 0.01-0.1 | 0.01-0.1 |
| Water | Q.S. | Q.S. | Q.S. | Q.S. | Q.S. | Q.S. |
| | to 100 | to 100 | to 100 | to 100 | to 100 | to 100 |
| | Ex. K | Ex. L | Ex. M | Ex. N | Ex. O | Comp. P |
| US INCI Name | (wt. %) | (wt. %) | (wt. %) | (wt. %) | (wt. %) | (wt. %) |
| Sodium Cocoyl Glycinate | 3.7-4.5 | 3.7-4.5 | 3.7-4.5 | 3.7-4.5 | 3.7-4.5 | 3.7-4.5 |
| Sodium Cocoyl | 0.7-1 | 0.7-1 | 0.7-1 | 0.7-1 | 0.7-1 | 0.7-1 |
| Hydrolyzed Collagen and | | | | | | |
| Glutamic Acid-N,N- | | | | | | |
| Diacetic Acid | | | | | | |
| Isopentane | 1 | 1 | 1 | 1 | 1 | 1 |
| Disiloxane | | | | | | |
| Dimethicone | 1.5 | | | | | |
| Coconut Acid | 0.4 | 0.9 | 1.4 | 1.9 | 0.4 | 0.4 |
| Tocopheryl Acetate | | | | | 0.5 | |
| Alpha-Tocopherol | x < | x < | x < | x < | x < | X < |
| | 0.001 | 0.001 | 0.001 | 0.001 | 0.001 | 0.001 |
| Isononyl Isononanoate and | 0.5 | 0.5 | 0.5 | 0.5 | 0.5 | 0.5 |
| Ethylhexyl Isononanoate | | | | | | |
| Polyhydroxystearic Acid | 0.5 | 0.5 | 0.5 | 0.5 | 0.5 | 0.5 |
| Cocamidopropyl Pg- | 0.15-3 | 0.15-3 | 0.15-3 | 0.15-3 | 0.15-3 | 0.15-3 |
| Dimonium Chloride | | | | | | |
| Phosphate | | | | | | |
| Phenoxyethanol, and | 1 | 1 | 1 | 1 | 1 | 1 |
| Ethylhexylglycerin | | | | | | |
| Glycerin | 1-3.5 | 1-3.5 | 1-3.5 | 1-3.5 | 1-3.5 | 1-3.5 |
| Hexylene Glycol, 1,3- | 0.2 | 0.2 | 0.2 | 0.2 | 0.2 | 0.2 |
| Propanediol, and Caprylyl | | | | | | |
| Glycol | | | | | | |
| Magnesium Silicate | 1-1.5 | 1-1.5 | 1-1.5 | 1-1.5 | 1-1.5 | 1-1.5 |
| Sodium Bisulfite, | 0.4-0.5 | 0.4-0.5 | 0.4-0.5 | 0.4-0.5 | 0.4-0.5 | 0.4-0.5 |
| Niacinamide, Licorice | | | | | | |
| Root Extract, and Sodium | | | | | | |
| PCA | | | | | | |
| Phenyl Propyl Alcohol | 0.01-0.1 | 0.01-0.1 | 0.01-0.1 | 0.01-0.1 | 0.01-0.1 | 0.01-0.1 |
| Acrylates Copolymer | 12-13 | 12-13 | 12-13 | 12-13 | 12-13 | 12-13 |
| Sodium hydroxide | 0.1-0.5 | 0.1-0.5 | 0.1-0.5 | 0.1-0.5 | 0.1-0.5 | 0.1-0.5 |
| Sodium chloride | 0.8-1.4 | 0.8-1.4 | 0.8-1.4 | 0.8-1.4 | 0.8-1.4 | 0.8-1.4 |
| Fragrance | 0.01-0.1 | 0.01-0.1 | 0.01-0.1 | 0.01-0.1 | 0.01-0.1 | 0.01-0.1 |
| Water | Q.S. | Q.S. | Q.S. | Q.S. | Q.S. | Q.S. |
| | to 100 | to 100 | to 100 | to 100 | to 100 | to 100 |

### Example 5

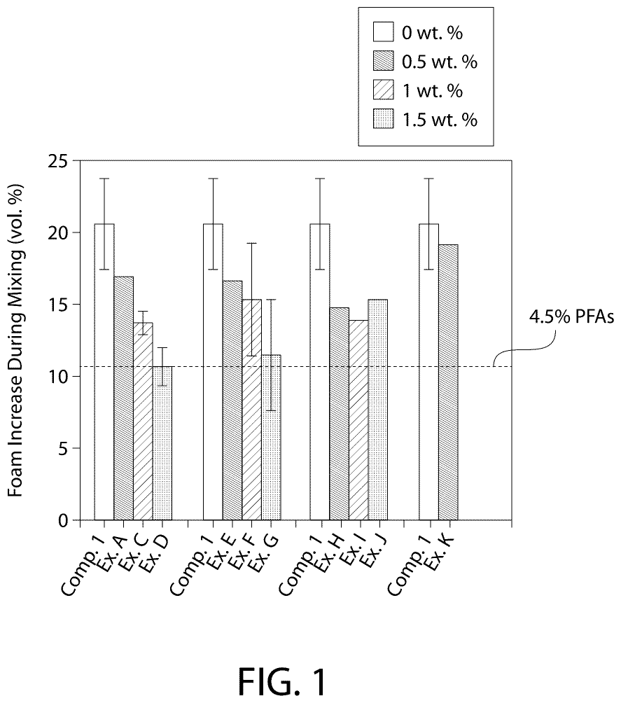

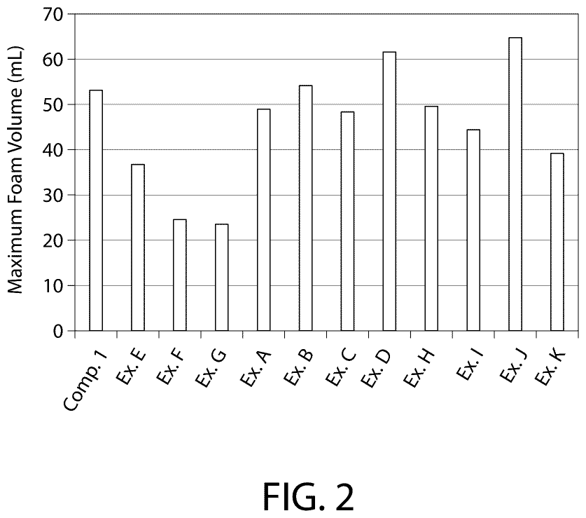

Example Compositions E-P and Comparative Composition 3 were evaluated during their production to assess the foam generation during their production process. Specifically, each personal care compositions was prepared by combining polyhydroxystearic acid, isononylisononanoate, ethylhexyl isononanoate, cocamidopropyl pg-dimonium chloride phosphate, phenyl propyl alcohol, caprylyl glycol, 1,3-propanediol, alpha-tocopherol, isopentane, deionized water, one of disiloxane, dimethicone, coconut acid, or tocopheryl acetate (if present) in the respective amount for such personal care composition to form a first mixture. The first mixture was then homogenized for 30 seconds at 3,800 to 4,000 rpm. The first mixture was then combined with a second mixture containing the remaining ingredients of the respective personal care composition and then mixed for 10 minutes at 50 rpm with overhead stirring. Each personal care composition was then left in the uncovered beaker for 30 minutes before measuring the foam height. FIGS. 1 and 2 are bar graphs showing the percent foam volume increase during mixing and the maximum foam height for Example Compositions E-P and Comparative Composition 3.

### Example 6

A non-limiting, exemplary self-foaming personal care composition (Example Composition Q) was prepared in accordance with aspects of the invention. The formulation for Example Composition L is provided in Table 6.

TABLE 6

| | Ex. Q |
|---|---|
| US INCI Name | (wt. %) |
| Acrylates Copolymer | 12-13 |
| Butylene Glycol | 1.7-2.7 |
| Cocamidopropyl Hydroxysultaine | 4.5-6 |
| Decyl Glucoside | 2.7-3.5 |
| Caprylyl/Capryl Glucoside | 2.7-3.5 |
| Sodium Cocoyl Hydrolyzed Collagen | 0.8 |
| and Glutamic Acid-N,N-Diacetic | |
| Acid | |
| Glycerin | 1.7-2.7 |
| Hexylene glycol, Caprylyl Glycol, | 0.2 |
| and 1,3-Propanediol | |
| Magnesium Silicate | 1-1.5 |
| Ethylhexyl Isononanoate and | 2 |
| Isononyl Isononanoate | |
| Polyhydroxystearic Acid | 0.5 |
| Cocamidopropyl PG-Dimonium | 0.2 |
| Chloride Phosphate | |
| Phenyl Propyl Alcohol | 0.01-0.1 |
| Alpha-Tocopherol | x < 0.001 |
| Isopentane | 1 |
| Niacinamide | 0.1-0.5 |
| Licorice Extract | 0.01-0.1 |
| Sodium PCA | 0.1-0.5 |
| Phenoxyethanol and | 1 |
| Ethylhexylglycerin | |
| Sodium Hydroxide | 0.1-0.5 |
| Fragrance | 0.01-0.1 |
| Water | Q.S. to |
| | 100 |

### Example 7

Two non-limiting, example self-foaming personal care compositions (Example Compositions R and S) were prepared in accordance with an aspect of the invention. The formulations for Example Compositions R and S are provided in Table 7.

TABLE 7

| | Ex. R | Ex. S |
|---|---|---|
| US INCI Compound Name | (wt. %) | (wt. %) |
| Sodium Cocoyl Glycinate | 1.6 | 4 |
| Sodium Cocoyl Apple Amino Acids | 0.8 | 0.8 |
| Sodium Cocoyl Isethionate | 3 | |
| Cocamidopropyl Betaine | 0.2 | 0.2 |
| Glycerin, Hexylene Glycol, Caprylyl | 2.7 | 2.7 |
| Glycol, and 1,3-Propanediol | | |
| Sodium L-Pyrrolidone Carboxylate | 0.4 | 0.4 |
| 3-Pyridinecarboxamide | 1 | 1 |
| Licorice Extract | 0.025 | 0.025 |
| Carbomer Polymer | 3.3 | 3.3 |
| Coconut Acid | 0.9 | 1.1 |
| Polyhydroxystearic Acid | 0.5 | 0.5 |
| Isononylisononanoate and Ethylhexyl | 0.5 | 0.5 |
| Isononanoate | | |
| Cocamidopropyl PG-Dimonium | 0.2 | 0.2 |
| Chloride phosphate | | |
| Dimethicone | 0.5 | 0.5 |
| Tocopheryl Acetate | 0.5 | 0.5 |
| Glycol Stearate and Glycol | 0.6 | 0.6 |
| Distearate | | |
| Phenoxyethanol, Ethylhexylglycerin, | 1.2 | 1.2 |
| Sodium Metabisulfite, Glutamic | | |
| Acid-N,N-Diacetic Acid, Phenyl | | |
| Propyl Alcohol, and Sodium | | |
| Benzoate | | |
| Sodium Hydroxide, Citric Acid, and | 0.8 | 1.2 |
| Sodium Chloride | | |
| Fragrance | 0.05 | 0.05 |
| Isopentane | 1 | 1 |
| n-Pentane 95% | 0.5 | 0.5 |
| DI Water | 61.2 | 54.4 |

### Example 81

Ten non-limiting, self-foaming personal care compositions (Compositions T-AC) were prepared in accordance with aspects of the disclosure. Other than the surfactant systems, contained therein, Compositions T-AC had substantially same formulations. The formulations for Compositions T-AC are shown in Table 8.

TABLE 8

| | Comp. | Comp. | Comp. | Comp. | Comp. |
|---|---|---|---|---|---|
| | T | U | V | W | X |
| US INCI Compound Name | (wt. %) | (wt. %) | (wt. %) | (wt. %) | (wt. %) |
| Sodium Cocoyl Glycinate | 4.2 | 1.2 | 1.2 | 1.2 | 1.2 |
| Sodium Cocoyl Apple Amino | 0.8 | 0.8 | 0.8 | 0.8 | 0.8 |
| Acid | | | | | |
| Sodium C₁₄₋₁₆ Olefin Sulfonate | | 3 | | | |
| Sodium Methyl Cocoyl Taurate | | | 3 | | |
| Disodium Cocoyl Glutamate | | | | 3 | |
| Sodium Cocoyl Isethionate | | | | | 3 |
| Tocopheryl Acetate | 0.5 | 0.5 | 0.5 | 0.5 | 0.5 |
| Carbopol Aqua Sf1 Polymer | 12.5 | 12.5 | 12.5 | 12.5 | 12.5 |
| Glycerin, Caprylyl Glycol, and | 2-2.5 | 2-2.5 | 2-2.5 | 2-2.5 | 2-2.5 |
| 1,3-Propanediol | | | | | |
| Sodium L-Pyrrolidone | 0.2-0.5 | 0.2-0.5 | 0.2-0.5 | 0.2-0.5 | 0.2-0.5 |
| Carboxylate | | | | | |
| 3-Pyridinecarboxamide | 0.9-1.2 | 0.9-1.2 | 0.9-1.2 | 0.9-1.2 | 0.9-1.2 |
| Hexamethyldisiloxane (0.65 Cst) | 0.4-0.7 | 0.4-0.7 | 0.4-0.7 | 0.4-0.7 | 0.4-0.7 |
| Phenoxyethanol, | 1-1.5 | 1-1.5 | 1-1.5 | 1-1.5 | 1-1.5 |
| Ethylhexylglycerin, Sodium | | | | | |
| Metabisulfite, and Phenyl Propyl | | | | | |
| Alcohol | | | | | |
| Coconut Acid | 0.5-1 | 0.5-1 | 0.5-1 | 0.5-1 | 0.5-1 |
| Polyhydroxystearic Acid | 0.5-1 | 0.5-1 | 0.5-1 | 0.5-1 | 0.5-1 |
| Isononylisononanoate and | 0.3-0.6 | 0.3-0.6 | 0.3-0.6 | 0.3-0.6 | 0.3-0.6 |
| Ethylhexyl Isononanoate | | | | | |
| Cocamidopropyl PG-Dimonium | 0.1-0.3 | 0.1-0.3 | 0.1-0.3 | 0.1-0.3 | 0.1-0.3 |
| Chloride phosphate | | | | | |
| Fragrance | 0.05 | 0.05 | 0.05 | 0.05 | 0.05 |
| Licorice Extract | 0.01-0.05 | 0.01-0.05 | 0.01-0.05 | 0.01-0.05 | 0.01-0.05 |
| iso-Pentane | 0.8-1.3 | 0.8-1.3 | 0.8-1.3 | 0.8-1.3 | 0.8-1.3 |
| | Comp. | Comp. | Comp. | Comp. | Comp. |
| | Y | Z | AA | AB | AC |
| US INCI Compound Name | (wt. %) | (wt. %) | (wt. %) | (wt. %) | (wt. %) |
| Sodium Cocoyl Glycinate | 1.2 | 1.2 | 1.2 | 1.2 | 1.2 |
| Sodium Cocoyl Apple Amino | 0.8 | 0.8 | 0.8 | 0.8 | 0.8 |
| Acid | | | | | |
| Cocamidopropyl Betaine | 3 | | | | |
| Cocamidopropyl | | 3 | | | |
| Hydroxysultaine | | | | | |
| Coco-Glucoside | | | 3 | | |
| Caprylyl/Capryl Glucoside | | | | 3 | |
| Sodium Lauroyl Sarcosinate | | | | | 3 |
| Tocopheryl Acetate | 0.5 | 0.5 | 0.5 | 0.5 | 0.5 |
| Carbopol Aqua Sf1 Polymer | 12.5 | 12.5 | 12.5 | 12.5 | 12.5 |
| Glycerin, Caprylyl Glycol, and | 2-2.5 | 2-2.5 | 2-2.5 | 2-2.5 | 2-2.5 |
| 1,3-Propanediol | | | | | |
| Sodium L-Pyrrolidone | 0.2-0.5 | 0.2-0.5 | 0.2-0.5 | 0.2-0.5 | 0.2-0.5 |
| Carboxylate | | | | | |
| 3-Pyridinecarboxamide | 0.9-1.2 | 0.9-1.2 | 0.9-1.2 | 0.9-1.2 | 0.9-1.2 |
| Hexamethyldisiloxane (0.65 Cst) | 0.4-0.7 | 0.4-0.7 | 0.4-0.7 | 0.4-0.7 | 0.4-0.7 |
| Phenoxyethanol, | 1-1.5 | 1-1.5 | 1-1.5 | 1-1.5 | 1-1.5 |
| Ethylhexylglycerin, Sodium | | | | | |
| Metabisulfite, and Phenyl Propyl | | | | | |
| Alcohol | | | | | |
| Coconut Acid | 0.5-1 | 0.5-1 | 0.5-1 | 0.5-1 | 0.5-1 |
| Polyhydroxystearic Acid | 0.5-1 | 0.5-1 | 0.5-1 | 0.5-1 | 0.5-1 |
| Isononylisononanoate and | 0.3-0.6 | 0.3-0.6 | 0.3-0.6 | 0.3-0.6 | 0.3-0.6 |
| Ethylhexyl Isononanoate | | | | | |
| Cocamidopropyl PG-Dimonium | 0.1-0.3 | 0.1-0.3 | 0.1-0.3 | 0.1-0.3 | 0.1-0.3 |
| Chloride phosphate | | | | | |
| Fragrance | 0.05 | 0.05 | 0.05 | 0.05 | 0.05 |
| Licorice Extract | 0.01-0.05 | 0.01-0.05 | 0.01-0.05 | 0.01-0.05 | 0.01-0.05 |
| iso-Pentane | 0.8-1.3 | 0.8-1.3 | 0.8-1.3 | 0.8-1.3 | 0.8-1.3 |

The self-foaming characteristic of Compositions T-AC was evaluated after accelerated aging of such personal care compositions. Specifically, samples of Compositions T-AC were placed in a controlled environment at a temperature of 40° C. for 4 weeks. Upon completion of the 4 weeks of accelerated aging, the samples of Compositions T-AC were evaluated to determine their rate of vertical foam growth and DFA lather performance.

To assess the rate of vertical foam growth, samples of Compositions T-AC were pumped into respective beakers. Subsequently, 1 mL of the respective personal care composition was taken from the beaker using a syringe and dispensed onto a heated substrate having a temperature of 37° C. The personal care composition was then rubbed in a circular motion 16 times at a pace of 96 rubs per minute, which is equivalent to 10 seconds. The foam growth of the personal care composition was video recorded in a photobox and analyzed using the in-house built software to assess the rate of vertical foam growth for Compositions T-AC.

To assess the DFA lather performance, samples of Compositions T-AC were diluted with water to produce a solution comprising 5 wt. % of the respective sample of Composition T-AC with the remainder being water. The DFA stirred the solution and measured the maximum foam height, which corresponds to the foam lathering of the personal care composition during use.

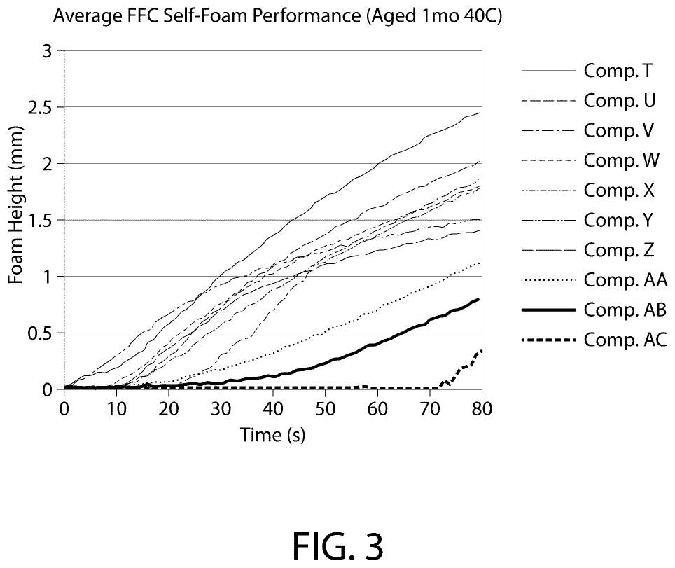

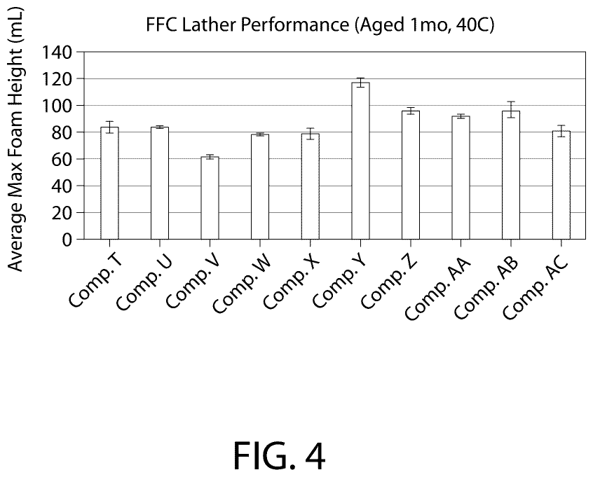

A summary of the rate of vertical foam growth as a function of time for Compositions T-AC is shown in FIG. 3. The average maximum foam height for Compositions T-AC is shown in FIG. 4.

### Example 9

A non-limiting, exemplary self-foaming personal care composition (Example Compositions AD) were prepared in accordance with an aspect of the invention. The formulation for Example Composition AD is provided in Table 9.

TABLE 9

| | Ex. AD |
|---|---|
| US INCI Compound Name | (wt. %) |
| Sodium Cocoyl Glycinate | 2.4 |
| Sodium Cocoyl Apple Amino Acids | 1.6 |
| Sodium Cocoyl Isethionate | 3 |
| Glycerin, Caprylyl Glycol, and 1,3- | 2.1 |
| Propanediol | |
| Sodium L-Pyrrolidone Carboxylate | 0.4 |
| 3-Pyridinecarboxamide | 1 |
| Licorice Extract | 0.03 |
| Carbomer Polymer | 3.2 |
| Coconut Acid | 1 |
| Polyhydroxystearic Acid | 0.5 |
| Isononylisononanoate | 0.2 |
| Ethylhexyl Isononanoate | 0.2 |
| Cocamidopropyl PG-Dimonium Chloride | 0.2 |
| phosphate | |
| Hexamethyldisiloxane (0.65 CST) | 0.5 |
| Tocopheryl Acetate | 0.5 |
| Cocamidopropyl Betaine | 0.2 |
| Glycol Distearate and Glycol Stearate | 0.6 |
| Phenoxyethanol, Ethylhexylglycerin, | 1.3 |
| Sodium Metabisulfite, Hexylene Glycol, | |
| Phenyl Propyl Alcohol, Sodium | |
| Benzoate, and Glutamic Acid-N,N- | |
| Diacetic Acid, Tetrasodium Salt | |
| Citric Acid | 0.01 |
| Sodium Hydroxide and Sodium Chloride | 1.1 |
| Fragrance | 0.05 |
| Isopentane | 1 |
| n-Pentane | 0.5 |
| DI Water | Q.S. to 100 |

### Example 10

A prophetic example of a self-foaming personal care composition (Example Composition AE) according to aspects of the invention is shown in Table 10.

TABLE 10

| | Ex. AD |
|---|---|
| US INCI Compound Name | (wt. %) |
| Sodium Cocoyl Glycinate | 2-3.3 |
| Sodium Cocoyl Apple Amino Acids | 1.4-2.1 |
| Sodium Cocoyl Isethionate | 2.8-3.5 |
| Glycerin, Caprylyl Glycol, and 1,3- | 1.8-2.3 |
| Propanediol | |
| Sodium L-Pyrrolidone Carboxylate | 0.3-0.6 |
| 3-Pyridinecarboxamide | 0.9-1.2 |
| Licorice Extract | 0.01-0.05 |
| Carbomer Polymer | 3-4 |
| Coconut Acid | 0.9-1.2 |
| Polyhydroxystearic Acid | 0.4-0.7 |
| Isononylisononanoate | 0.1-0.3 |
| Ethylhexyl Isononanoate | 0.1-0.3 |
| Cocamidopropyl PG-Dimonium | 0.1-0.3 |
| Chloride phosphate | |
| Hexamethyldisiloxane (0.65) CST | 0.4-0.7 |
| Tocopheryl Acetate | 0.4-0.7 |
| Cocamidopropyl Betaine | 0.1-0.3 |
| Glycol Distearate and Glycol Stearate | 0.5-0.8 |
| Phenoxyethanol, Ethylhexylglycerin, | 1-1.5 |
| Sodium Metabisulfite, Hexylene | |
| Glycol, Phenyl Propyl Alcohol, and | |
| Sodium Benzoate | |
| Citric Acid | 0.01 |
| Sodium Hydroxide and Sodium | 1.1 |
| Chloride | |
| Fragrance | 0.05 |
| Isopentane | 1 |
| n-Pentane | 0.5 |
| DI Water | Q.S. to 100 |

What is claimed is:

1. A personal care composition comprising:
a hydrocarbon having 4 to 6 carbons;
a fatty ester, a fatty acid, a medium chain triglyceride, a silicone, vitamin E, or a combination of two or more thereof; and
a surfactant system,
wherein the personal care composition is self-foaming.

2. The personal care composition according to claim 1, wherein the hydrocarbon having 4 to 6 carbons comprises pentane, isopentane, or a combination thereof.

3. The personal care composition according to claim 2, wherein the personal care composition has a weight ratio of isopentane to pentane of about 4:1 to about 1:3.

4. The personal care composition according to claim 1 comprising a fatty ester, wherein the fatty ester is selected from the group consisting of cetyl ester, cetearyl octanoate, isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, octyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, dicaprylyl carbonate, pentaerythritol esters, polyglyceryl-10 oleate, polyglyceryl-10 dioleate, polyglyceryl-6 stearate, polyglyceryl-6 distearate, polyglyceryl-10 stearate, polyglyceryl-10 distearate, polyglyceryl-8 dipalmitate, polyglyceryl-10 dipalmitate, polyglyceryl-10 behenate, polyglyceryl-12 trilaurate, ethylhexyl isononanoate, and a combination of two or more thereof.

5. The personal care composition according to claim 1 comprising a fatty acid, wherein the fatty acid comprises octanoic acid, lauric acid, palmitic acid, stearic acid, behenic acid, arachidonic acid, oleic acid, isostearic acid, polyhydroxystearic acid, sebacic acid, coconut acid, or a combination of two or more thereof.

6. The personal care composition according to claim 1, wherein the personal care composition has a weight ratio of the total amount of the hydrocarbon having 4 to 6 carbons to the total amount of the fatty ester, the fatty acid, or the combination thereof of about 2:1 to about 1:7.

7. The personal care composition according to claim 1 comprising a silicone, wherein the silicone is selected from the group consisting of disiloxane, polydimethicone, dimethiconol, amodimethicone, aminoethylaminopropyl dimethicone, aminopropyl dimethicone, trimethylsilylamodimethicone, trisiloxane, tetrasiloxane, and a combination of two or more thereof.

8. The personal care composition according to claim 1, wherein the personal care composition has a weight ratio of the total amount of the hydrocarbon having 4 to 6 carbons to the total amount of the silicone of about 4:1 to about 1:5.

9. The personal care composition according to claim 1, wherein the personal care composition has a weight ratio of the total amount of the hydrocarbon having 4 to 6 carbons to the total amount of the vitamin E of about 8:1 to about 1:1.

10. The personal care composition according to claim 1, wherein the personal care composition is substantially free of or free of triglycerides other than medium chain triglycerides.

11. The personal care composition according to claim 1, wherein the surfactant system comprises an acyl amino acid surfactant that is derived from alanine, arginine, aspartic acid, glutamic acid, glycine, isoleucine, leucine, lysine, phenylalanine, serine, tyrosine, valine, sarcosine, threonine, or taurine.

12. The personal care composition according to claim 1, wherein the surfactant system comprises an anionic surfactant of plant protein, wherein the anionic surfactant of plant protein is selected from the group consisting of sodium cocoyl hydrolyzed keratin, sodium cocoyl hydrolyzed rice protein, sodium cocoyl apple amino acid, sodium cocoyl hydrolyzed soy protein, sodium cocoyl hydrolyzed sweet almond protein, sodium cocoyl hydrolyzed wheat protein, sodium cocoyl hydrolyzed wheat protein glutamate, sodium cocoyl oat amino acids, sodium cocoyl/palmoyl/sunfloweroyl glutamate, potassium lauroyl wheat amino acid, sodium lauroyl wheat amino acid, sodium lauroyl oat amino acid, potassium undecylenoyl hydrolyzed soy protein, sodium undecylenoyl hydrolyzed soy protein, sodium dilauramidoglutamide lysine, sodium cocoyl threoninate, sodium cocoyl glutamate, disodium cocoyl glutamate, and a combination of two or more thereof.

13. The personal care composition according to claim 1, wherein the surfactant system comprises an anionic surfactant of silk protein.

14. The personal care composition according to claim 1, wherein the surfactant system comprises a nonionic surfactant, the nonionic surfactant comprising a lactylate surfactant.

15. The personal care composition according to claim 14, wherein the lactylate surfactant is selected from sodium decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, and a combination of two or more thereof.

16. The personal care composition according to claim 1, wherein the surfactant system comprises a nonionic surfactant, the nonionic surfactant comprising an alkyl glucoside selected from the group consisting of decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination of two or more thereof.

17. The personal care composition according to claim 1, wherein the surfactant system comprises sodium cocoyl glycinate in an amount from about 0.5 to about 6 wt. %, based on the total weight of the personal care composition.

18. The personal care composition according to claim 1, wherein the surfactant system comprises a total amount of sodium cocoyl glycinate and/or potassium cocoyl glycinate of about 1.6 to about 5 wt. %, and at least one surfactant selected from the group consisting of an acyl amino acid surfactant other than sodium cocoyl glycinate or potassium cocoyl glycinate, a sulfonate surfactant, an anionic taurate surfactant, an anionic glutamate surfactant, an anionic isethionate surfactant, a betaine surfactant, a glucoside surfactant, and a combination of two or more thereof.

19. The personal care composition according to claim 1, wherein the personal care composition is formulated to produce a first foam height of about 1 mm or more within 30 seconds after applied to a heated substrate at a temperature of about 37° C. and rubbed in a circular motion about 16 times at a rate of 96 rubs per minute and produce a second foam height within 80 seconds of said being rubbed in a circular motion, wherein the second foam height is at least 45% greater than the first foam height.

20. A personal care product comprising:
a container containing a personal care composition, the personal care composition comprising:
a hydrocarbon having 4 to 6 carbons;
a fatty ester, a fatty acid, a medium chain triglyceride, a silicone, vitamin E, or a combination of two or more thereof; and
a surfactant system,
wherein the personal care composition is self-foaming, and
wherein the personal care product is not pressurized.
